SEQUENCE LISTING (15 sequences)

SEQ ID NO: 1 - DNA, 549 bp, Oryza sativa
atcagcagcagccacagcaaaatttggtttgggataggtaggtgttatgttaggtctggttttttggctgtagcagcagcagtggaaggggcatgcagaggagcaggagattcagtttgaagctggacttcacttttgcctctctctcctgtgcttgcctcttccattcctgctgctaggctgttctgtggaagtttgcagagtttatattatgggtttaatcgtccatggcatcagcatcagcagcggtaggagaaacttttctgttattgcaccaaactctcaacatttcggtcattctgctccaggtctgaattcatatccatttcactaattctgtctacaggtatgtcatgacaatgcggccgtgcgagctccatgagctcgaatgccattgcagtggattcaggacaacgaaatggacactgtttttcgttggaaatttgggcgtatgccgttgtaaaattcatgtttcagagaaatgccgctacaaaatgtatctatgagcttgaagttatctcaaagctatcagtaacacattatttggtc

SEQ ID NO: 2 - RNA, 88 nt, Oryza sativa
aguggaaggggcaugcagaggagcaggagauucaguuugaagcuggacuucacuuuugccucucucuccugugcuugccucuuccauu

SEQ ID NO: 3 - DNA, 348 bp, Agrostis stolonifera
ggcgagccccagacgatcctcatcaacgggagagggcagtacgactgcatgctggggaaggtgacgaactaccacaggggcatcgacgggtacgccaagacctgcgtgaggggcaaggaggccaagctgtgcaatgaccaggagcggtgcgtgaggcgaagcgagtgcggtccctactgccccgagagccagtgcgcgcccgtggtcttcgacgtggagcctggcaagacgtaccggctcaggatcgccagcaccacctccctctctgctctcaacctgcaggtcgaagggcacgagctgacggtggtggaggcggacggcaacccggtggagccattcaaggtcacg

SEQ ID NO: 4 - DNA, 2191 bp, Oryza sativa
agtgtagcatagctcacttccaagagcctcgtgacacacaccactcactatactggcccctcctcctcctcggagatcgagccggccagccatggcggccgccgtgcagctgctcgtcgtcgccgccgccgccgccatggcggcggcgtgctgcgccggcatgacggcggcggcggcgacggtggaggtgacgtgggacgtggagtacgtactgtgggcgccggactgccagcagcgggtgatgatcgggataaacggcaggttcccggggcccaacatcaccgcgcgcgccggcgacgtgatcagcgtcaccatgaacaacaagatgcacaccgagggcgtcgtcatccactggcacggcatcagacagtttggcacgccgtgggcggacgggacggcatcgatatcccagtgcgcagtgaacccgggcgagacgttcgtctacaagttcgtcgccgacaagccgggcacctacttctaccacggccacttcgggatgcagcgcgccgcgggcctgtacggttccctcatcgtcctcgactcgccggagcagcccgagccgttccgccaccagtacgacgacggcggcgagctccccatgatgctcctcagcgactggtggcaccagaacgtctacgcccaggccgccggactcgacggcaaggacaggcacttcgagtggatcggcgagccccagacgatcttgatcaatgggagaggacagttcgagtgcacgctggggccagcgaggaagagctttgagaagctcctcaacgagaacgtggagacctgcgtcgacgaccagaagatgtgcagcgaccaggagaagtgcctgaggaggagcgagtgcgggccgtactgccccaggagccagtgcgcccctgtcgtgttcaatgtcgagcaggggaagacttaccgccttaggatcgccagcaccacctccctttctctcctcaacgtcaagattcaagggcacaagatgacggtggtggaggccgacgggaaccacgtggagccgttcgtggtcgacgacatcgacatctactccggcgagagctactccgtcctcctcaaggccgaccagaagccggcgagctactggatctccgtcggcgtcagggggcgccaccccaagacggtgccggcgctcgccatcctcagctacggcaacggcaacgcggcgccgccgccgctccagctgcccgccggcgagccccccgtgacgccggcgtggaacgacacacagcgcagcaaggccttcacctacagcatcagggcgcgcaaggacaccaaccggccgccgccggcggccgccgaccggcagatcgtcctgctcaacacgcagaacctcatggacgggcgctacaggtggtccatcaacaacgtgtccctgacgctgccggcgacgccgtacctgggcgccttccaccacggcctccaggacagcgcgttcgacgcgtccggcgagccgccggcggcgttcccggaggactacgacgtgatgaggccgccggcgaacaacgcgacgacggcgagcgacagggtgttccggctgcgacacggcggcgtggtggacgtggtgctccagaacgccaacatgctgagggaggaggtgagcgagacgcacccgtggcacctccacggccacgacttctgggtgctcggctacggcgacggccggtacgacccggcggcgcacgcggcggggctcaacgccgccgacccgccgctgcggaacacggcggtggtcttcccgcacgggtggacggcgcttcggttcgtcgccaacaacaccggcgcgtgggcgttccactgccacatcgagccgcacctccacatgggcatgggcgtcgtcttcgtcgagggggaggacaggatgcacgagctcgacgtgcccaaggacgccatggcgtgcggcctcgtcgccaggacggccgccacgccgctcaccccggcaacgccgctgcctccgtcgccggcgccggcgccatgagctcctcctcagcatgcccattccagttaaatgccatttttgccgtaacattgtgattggccactgcgaaataagatcactcactgatgaagagtggtttagattgtttggtcatatgcatgctcaataatctcatgtaactaagaaataatgtccatgtttgctaattaagatgattgtgtaatattgttgcaatgt

SEQ ID NO: 5 - DNA, 2059 bp, Arabidopsis thaliana
ccacttttgtttttaaggttcaatttttgaaattttcaaaaaacaaagcctatttcatacactcaccagtgcttcacaattcatcataattcataagccacgcaaaagaaaataaagtgaaagattccatggcggtaattgtgtggtggctactaacggtggttgtggtggcgtttcactcggcgtcggcggcagtagtagaatcaacgtgggaggttgagtataagtactggtggccggattgtaaagaaggaatcgttatggccatcaacggccagtttccagggccaacgatagacgcagtcgccggagacacggtcattatccacgtcgtcaacaaactctccaccgaaggtgttgttatccattggcacggcatacgtcagaaagggactccatgggctgatggagcagcaggtgtgacccagtgtcctattaatcctggcgagactttcacttacaagttcattgtcgataaggcgggaacacatttctaccatggacactacggaatgcaaagatcatcgggactatacggaatgttaatagtaagatcaccaaaagagaggttaatttacgatggagagttcaatctcttgctcagtgactggtggcaccaaagcattcacgcgcaagaactcgctctttcttctcgtcctatgcgttggatcggtgaacctcaaagcttgttgatcaatggaagaggacagttcaattgttcacaagcagcgtattttaacaaaggaggagagaaagatgtatgcacgtttaaagaaaatgatcagtgtgcacctcaaactcttcgagtcgaacccaatagagtgtaccgtcttcgaatcgctagcacaactgctcttgcttccctcaacttggctgttcaaggacaccagctggtggttgttgaagctgacggcaactacgtcgcaccgttcaccgtcaacgacattgacgtttattccggcgaaacttattctgttctccttaaaaccaacgcacttccatcaaagaagtactggatctccgtcggcgttcgtggccgagaacccaaaactcctcaagcactcaccgtgataaattacgttgatgccactgagtcacgcccatctcatccaccaccggtgactccaatctggaacgacacagatcggagcaaaagcttctcgaagaagatcttcgccgctaaaggatatccaaaaccgccggagaaatcacatgaccagctaatcctcctcaacacacagaatctctacgaagattacacgaaatggtcaatcaacaacgtctcattatccgtaccggtgacgccgtacctcggatcaattagatacggtttaaaatcggcgtacgatttgaaatcgccggcgaagaagttaattatggataattacgatatcatgaaaccgccgccgaatccaaacacaacgaaaggtagcgggatttacaatttcgcgtttggaatcgtcgtcgacgtgattcttcaaaacgctaatgttttaaaaggtgtgattagtgagattcatccgtggcatattcatggtcatgatttctgggttttgggttacggtgaagggaaatttaaaccggggattgatgagaagacgtttaatttgaagaatccgccgttacggaatacggtggtgttgtatccgtttggatggacggcgataaggtttgtgacggataatccaggggtttggttttttcattgtcatattgaaccgcatttgcatatgggtatgggtgtggttttcgtggagggagtagaccggattggtaagatggagataccggatgaagcgcttggttgtggattaaccaggaaatggcttatgaaccggggacgcccttaaatgaaccggggttggtatgtgtgtttattttcctcgttgggtttcttaattttttaaagggataaaaataaggatgttctaattgataggatcaaagaaaatatgtttaattctcttgttgggggacaagatctctttctaaattatttttatggtgcaacaggatctcttaagtgtatgatttattaaagaaacttaaaatcttattt

SEQ ID NO: 6 - DNA, 385 bp, Agrostis stolonifera
acggtgaaggctgttgctgtgcttttggggcagtgcgggtgtcacgggcaccatctttttcacccaggagggagatggtccgaccaccgtgacaggaagcgtctctggactcaaggaaggcctccacggcttccatgtgcacgctcttggtgacaccactaatggctgcatgtcaactggaccgcacttcaaccccgctggtcatgtgcatggggcaccagaagatgaaatccgccatgctggagatcttggaaatgtgacagctggagtggatggtgttgctaccatcactgttgttgacaaacatattcccctttgtggaccacattcaatcattggccgtgctgttgctgtccatggtgatcctgatgatcttggcaagggt

SEQ ID NO: 7 - DNA, 828 bp, Oryza sativa
agaagctccagattccaaaccagcaggagtcgcctcgcctcctccttcatcctcctcgtcgtcgccgcgggggtcgcctgagatcacattaacaatggtgaaggctgttgttgtgcttggtagcagtgagattgttaagggcactatccactttgtccaagagggagatggtcccaccactgtgactggaagtgtctctggcctcaagcctggtctccatgggttccatattcatgcacttggtgacaccaccaatggttgcatgtcaactgggccacactacaatcctgccggaaaggagcatggagcaccagaagatgagacccgccatgctggtgatcttggaaatgtcaccgctggagaagatggtgttgctaatatccatgttgttgacagtcagattccacttactggaccaaattcaatcattggcagagccgtcgttgtgcatgccgatcctgatgatcttggaaagggtgggcacgagctgagcaagaccaccggaaacgctggtggccgtgttgcttgcgggatcatcggacttcaaggctgaaacctggaggtgtgaactcaccttccatctcccagcaccagaagcctgaaactctacgagctcttagccgtttcgtctttacctgagtggctactctagattctacaataagcacctgatctctgcgcatggtttttggtgtaccattctgtcgcccgcatcgttggcgcccaatgaactatgtgttttgtgttaaaccttaagctgaagggtaccatttgtagactcgatgttgctctcttcctcggaatgtcgttacatctgttcgctcgttt

SEQ ID NO: 8 - DNA, 788 bp, Arabidopsis thaliana
aagaagagtgagtgaagcaaaaacattcagagagaaaattcagcatttttgatagctcaagcacttgattctttccaaaggggtttcctgagatcacaaaggccaagtaacaatggcgaaaggagttgcagttttgaacagcagtgagggtgttacggggactatctttttcacccaggaaggcgatggtgtgaccactgtgagtggaacagtttctggccttaagcctggtcttcatggtttccatgtccatgctcttggtgacaccactaacggttgcatgtctactggtccacatttcaaccccgatggtaaaacacacggtgcccctgaggatgctaatcgacatgctggtgatctaggaaacatcactgttggagatgatggaactgccaccttcacaatcactgattgccagattcctcttactggaccaaactctattgttggtagggctgttgttgtccatgcagaccctgatgacctcggaaagggaggccatgaactcagcctggctactggaaacgcaggcggccgtgttgcttgcggcatcattggtctccagggctaaagctgctacgtttccaaagaagagattgatgtaataaggaggtccaaccttagacctgggtttggtagttgtgtgtatcttctggtgtgtggctaaaaacctatgagcttagtgtggctcaaagcatttttaattcagacagaaaacagagaaaattccgtacttttattatttcatgaataaaaaagagttgatttactgttaaaaaaaaaaaaaa

SEQ ID NO: 9 - DNA, 1038 bp, Oryza sativa
aagctaagctaaaccttatcatcgtcaggtcaggcacttcaccaaccctctgcctcccttccgccgccctccgccgccgcatgcaagccatcctcgccgctgccatggccgcccagaccctcttgttctccgccaccgcccctcccgcctcccttttccagtccccttcctctgcccgccctttccactcgctccgcctcgccgccggccccgcgggcgccgccgctgccagggcgctcgtcgtcgccgacgccaccaagaaggccgtcgccgtgctcaagggcacctcccaggttgagggagtcgtcaccctcacccaggatgaccaaggtcctacaacagtgaatgtccgtgtgacgggacttactcctggacttcacggcttccacctccacgagtttggcgatactacgaatgggtgcatatcaacaggaccacattttaacccaaacaatttgacgcacggtgcaccagaagatgaagtccgtcatgcgggtgacctgggaaacattgttgccaatgctgaaggtgtagctgaggcaaccattgttgataagcagattcctctgagtggcccaaattctgttgttgggagagcattcgttgttcatgagcttgaagatgatttggggaagggtggccatgagcttagtctcagtactggaaatgctggtgggcgacttgcatgcggtgttgttgggctgaccccgttgtaggtcgctgcaagttgcagctgaagtgtcagtatcgcatccatgtcaccctttttgtcatcttcgagcctgaggcagtcgttcttgtatcacatggatttcgcaacatggatgcttaatagtatctgttgatcgttcgtctcacagtaataaaatttagttgagcaaataagtgtcgtcacatcccctgttctccaccctgtcaaactataaattgtgaaacatgagctgttctgggtatacaacgcataaaaaaaaccatgtgttatacataacagccttgttgctggctgtactttcgtgcttctgatctaatctttgtgct

SEQ ID NO: 10 - DNA, 700 bp, Arabidopsis thaliana
aagtatttgtgcctactcatttcctccaaacgtcaaacatagcagcagccatggctgccaccaacacaatcctcgcattctcatctccttctcgtcttctcattcctccttcctccaatccttcaactctccgttcctctttccgcggcgtctctctcaacaacaacaatctccaccgtctccaatctgtttccttcgccgttaaagctccgtcgaaagcgttgacagttgtttccgcggcgaagaaggctgttgcagtgcttaaaggtacttctgatgtcgaaggagttgttactttgacccaagatgactcaggtcctacaactgtgaatgttcgtatcactggtctcactccagggcctcatggatttcatctccatgagtttggtgatacaactaatggatgtatctcaacaggaccacatttcaaccctaacaacatgacacacggagctccagaagatgagtgccgtcatgcgggtgacctgggaaacataaatgccaatgccgatggcgtggcagaaacaacaatagtggacaatcagattcctctgactggtcctaattctgttgttggaagagcctttgtggttcacgagcttaaggatgacctcggaaagggtggccatgagcttagtctgaccactggaaacgcaggcgggagattggcatgtggtgtgattggcttgacgccgctcta

SEQ ID NO: 11 - DNA, 23 bp, Artificial Sequence
  Description of Artificial Sequence Synthetic primer
tctagagatcagcagcagccaca

SEQ ID NO: 12 - DNA, 26 bp, Artificial Sequence
  Description of Artificial Sequence Synthetic primer
gtcgacgaccaaataatgtgttactg

SEQ ID NO: 13 - DNA, 50 bp, Artificial Sequence
  Description of Artificial Sequence Synthetic primer
gtcgtatccagtgcagggtccgaggtattcgcactggatacgacctcctc

SEQ ID NO: 14 - DNA, 19 bp, Artificial Sequence
  Description of Artificial Sequence Synthetic primer
gcagtggaaggggcatgca

SEQ ID NO: 15 - protein, 4 aa, Unknown
  Description of Unknown WRKY family motif peptide
WRKY

(19) United States
(12) Patent Application Publication
(10) Pub. No.: US 2015/0337328 A1
(43) Pub. Date: Nov. 26, 2015

(54) Methods and Constructs for Conferring Enhanced Abiotic Stress Resistance in Plants

(71) Applicant: CLEMSON UNIVERSITY, Clemson, SC (US)

(72) Inventors: Hong Luo, Clemson, SC (US); Zhigang Li, Clemson, SC (US); Shuangrong Yuan, Pendleton, SC (US); Qian Hu, Clemson, SC (US)

(21) Appl. No.: 14/719,659

(22) Filed: May 22, 2015

Related U.S. Application Data
(60) Provisional application No. 62/002,553, filed on May 23, 2014.

(51) Int. Cl. C12N 15/82

(52) U.S. Cl. CPC C12N 15/8273; C12N 15/8271

(57) ABSTRACT

A conserved monocot-specific miRNA, miR528, is described that can be utilized for mediating multiple stress responses and/or mediating morphological aspects of plant development. Also described are transgenic plant cells, plant parts such as seeds and plants as well as progeny of the seeds and plants that include a recombinant polynucleotide including a nucleic acid molecule encoding miR528. Also disclosed are targets of miR528, all of which appear to function in oxidation-reduction processes.

### CROSS REFERENCE TO RELATED APPLICATION

This application claims filing benefit of U.S. Provisional Patent Application Ser. No. 62/002,553, having a filing date of May 23, 2014, which is incorporated herein by reference in its entirety.

### STATEMENT AS TO RIGHTS TO INVENTIONS MADE UNDER FEDERALLY SPONSORED RESEARCH

This invention was made with Government support under competitive grant no. 2010-33522-21656 awarded by the United States Department of Agriculture's National Institute of Food and Agriculture under the Biotechnology Risk Assessment Grant Program and under grant no. CSREES SC-1700450 awarded by the United States Department of Agriculture. The Government has certain rights in the invention.

### SEQUENCE LISTING

The instant application contains a Sequence Listing which has been submitted electronically in ASCII format and is hereby incorporated by reference in its entirety. Said ASCII copy, created on May 19, 2015, is named CXU-818_SL.txt and is 14,196 bytes in size.

### BACKGROUND

Abiotic stresses are environmental stresses that restrict growth and/or productivity of plants. Abiotic stresses affect almost every aspect of plant life-cycle, including morphological, physiological, biochemical and molecular processes. Notable abiotic stresses include extremes in temperature, light or other radiation, water availability, and salt levels. Drought is the most pervasive abiotic stress and initiates from water deficits due to any number or reasons. Moreover, water deficits often lead to salt stress because of insufficient precipitation for soil leaching when the soil salinity is high. Thus, both drought and salt stresses are considered as water stress. Another common abiotic stress is nitrogen deficiency resulting from insufficient nitrogen supply in the soil.

To enhance plants' abiotic stress tolerance, both conventional breeding and genetic engineering methods have been adopted. Many genes encoding for particular functional proteins, transcription factors, and proteins involved in signaling pathways have been identified as drought, salt or nitrogen responsive genes. Plants subject to drought and salt stresses have been engineered to induce expression of genes encoding for late embryogenesis abundant (LEA) proteins, enzymes for osmolyte biosynthesis, molecular chaperones, antioxidative enzymes, protein kinases, enzymes for ABA biosynthesis, as well as transcription factors from the families of DREB, NAC, WRKY (SEQ ID NO: 15), MYB and MYC. Expression of such genes can enhance plant salt and/or drought tolerance. To improve plant performance under nitrogen deficiency conditions, substantial efforts have concentrated on understanding the physiological and molecular process of plant nitrogen use efficiency (NUE) which includes nitrogen uptake, assimilation, translocation, and remobilization. To improve NUE, a large number of crop plants have been genetically engineered by single functional genes involved in molecular pathways of NUE steps, but the success is limited due to the post-transcriptional regulation.

One of the adaptive mechanisms that plants have evolved in stress response is mediated by microRNAs (miRNAs). miRNAs are small regulatory noncoding RNAs with the length of approximately 19-24 nucleotides. They are a class of noncoding small RNAs that originate from precursor pri-miRNA transcripts that are encoded by endogenous miRNA genes. The pri-miRNA transcripts are processed to form the final miRNA that can regulate expression. They exert their function via imperfect complementary binding to their target mRNAs to induce transcriptional cleavage or translational inhibition. To date, increasing evidence suggests that plant miRNAs play important roles in response to various abiotic stresses as well as in regulation of plant morphology. For example, constitutive expression of miR396, which controls plant cell proliferation and division by targeting transcripts from growth-regulating factor (GRFs) family, leads to reduced leaf size in Arabidopsis as well as reduced salt and alkali tolerance in rice.

What are needed in the art are additional materials and methods for regulating abiotic stress response in plants.

### SUMMARY

According to one embodiment, disclosed a transgenic plant cell including a recombinant nucleic acid molecule, the recombinant nucleic acid molecule comprises a polynucleotide encoding miR528 operatively associated with a promoter. For instance, the nucleic acid molecule can include SEQ ID NO: 1 or SEQ ID NO: 2.

Also disclosed is a transgenic plant or progeny thereof or a transgenic seed or progeny thereof including the nucleic acid molecule that comprises a polynucleotide encoding miR528 operatively associated with a promoter.

Also disclosed is a method for producing a plant having increased tolerance to abiotic stress. More specifically, a method can include transforming a plant cell with a recombinant nucleic acid molecule that includes a nucleotide that encodes miR528 operatively associated with a primer and generating a plant from the transformed plant cell.

### BRIEF DESCRIPTION OF THE FIGURES

The present disclosure may be better understood with reference to the figures including:

FIG. 1A presents stem-loop RT-qPCR relative expression analyses of miR528 mature sequence in WT creeping bentgrass under 200 mM salt treatment.

FIG. 1B presents stem-loop RT-qPCR relative expression analyses of miR528 mature sequence in WT creeping bentgrass under drought treatment.

FIG. 1C presents stem-loop RT-qPCR relative expression analyses of miR528 mature sequence in WT creeping bentgrass under nitrogen deficiency.

FIG. 2A presents a schematic diagram of an Osa-miR528 gene overexpression construct (p35S-Osa-miR528/p35S-Hyg) the Osa-miR528 gene is under the control of Cauliflower Mosaic Virus (CaMV) 35S promoter and linked to the hygromycin resistance gene, Hyg, driven by CaMV 35S promoter (RB: right border; LB: left border).

FIG. 2B illustrates the PCR analysis to amplify hyg gene in genomic DNA of transgenic (TG) and wild-type (WT) creeping bentgrass to determine the integration of Osa-miR528 gene in the host genome.

FIG. 2C illustrates the results of real-time RT-PCR analysis to detect the expression of primary Osa-miR528 in the transcripts of TG and WT plants.

FIG. 2D presents the stem-loop RT-qPCR analysis to detect the expression of mature Osa-miR528 in the transcripts of TG and WT plants.

FIG. 3A illustrates ten-week-old WT and TG plants initiated from a single tiller. Scale bar, 10 cm.

FIG. 3B illustrates two-month-old WT and TG plants initiated from the same amount of tillers were grown in the same 6-inch pot. Scale bar, 10 cm.

FIG. 3C illustrates a close up of the longest tillers from WT and TG plants, respectively. Scale bar, 5 cm.

FIG. 3D illustrates all internodes from the representative longest tiller were sliced from top to bottom and arranged from left to right. Scale bar, 5 cm.

FIG. 3E illustrates the top three fully developed leaves from the representative tillers of WT and TG plants. Scale bar, 2 cm.

FIG. 3F includes cross section images of WT and TG leaves. Scale bar, 200 μm.

FIG. 3G includes cross-section images of WT and TG stems. Scale bar, 100 μm.

FIG. 3H is a statistical analysis of leaf thickness between representative WT and TG plants (n=8).

FIG. 3I is a statistical analysis of the number of vascular bundles between representative WT and TG stems (n=8).

FIG. 4A illustrates tiller number in WT and TG plants 5&10-week after initiation from a single tiller (n=5).

FIG. 48 illustrates the total shoot number including both tiller and lateral shoot number in WT and TG plants 30, 60, and 90-day after initiation from a single tiller (n=5).

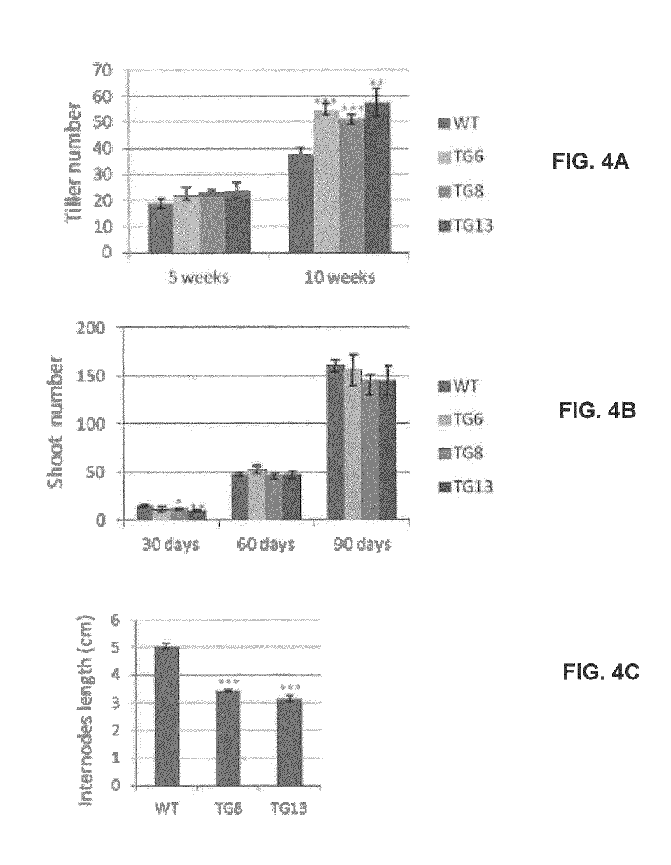

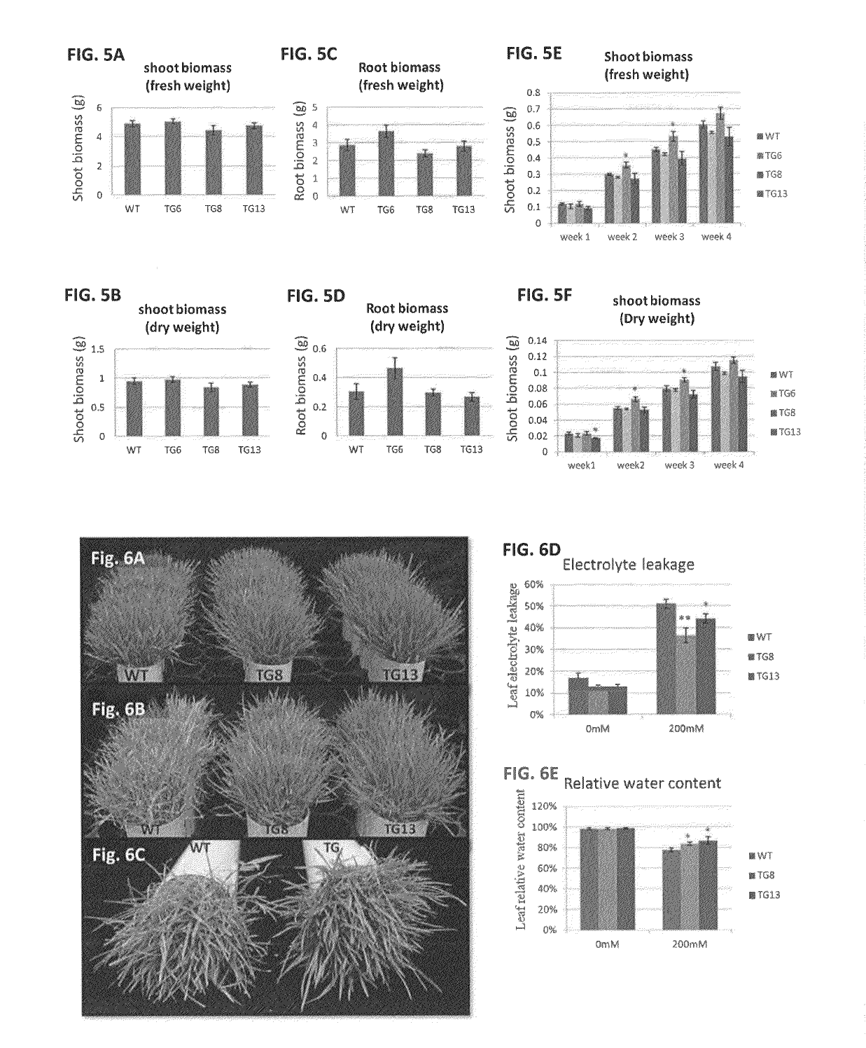

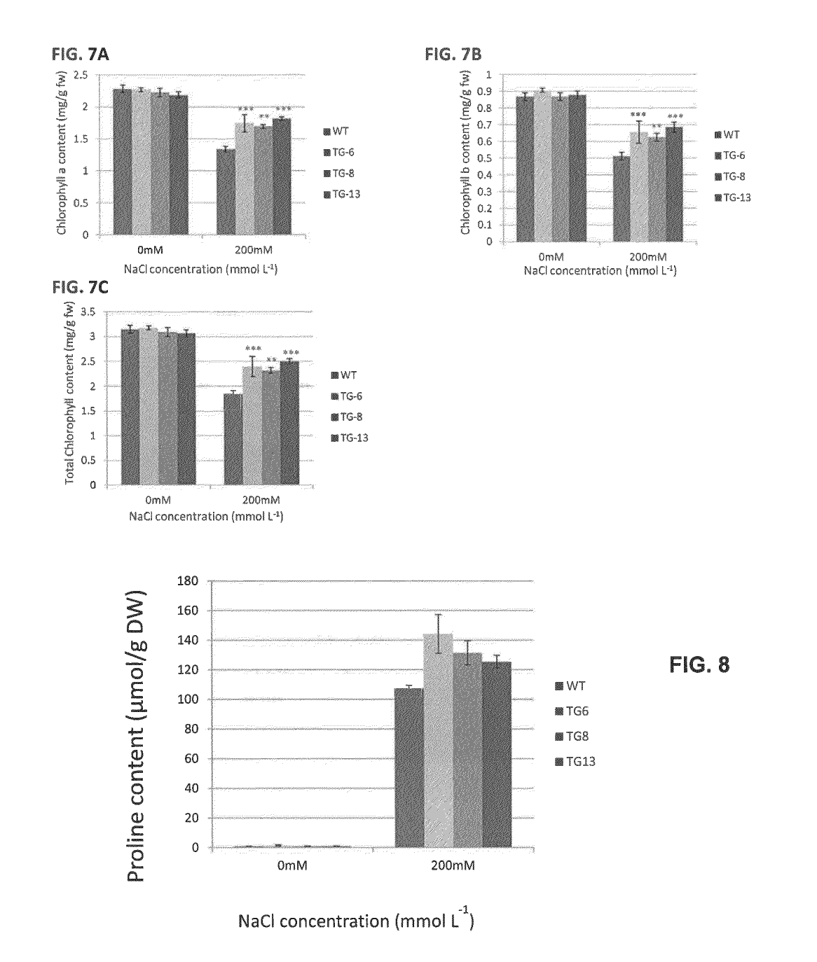

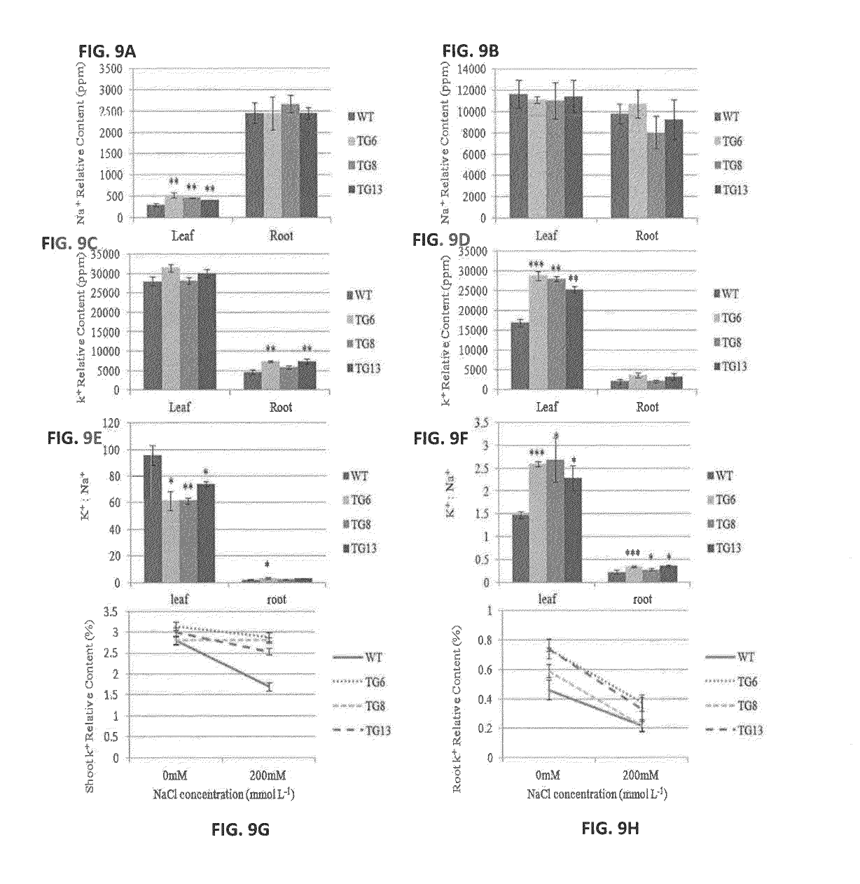

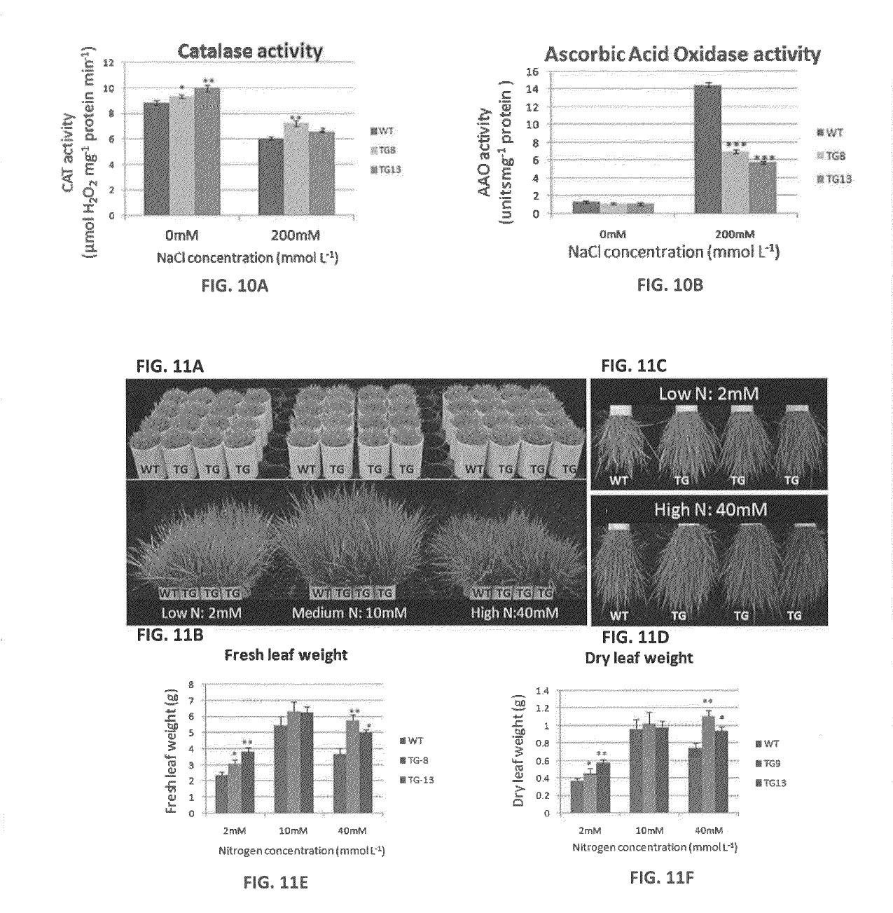

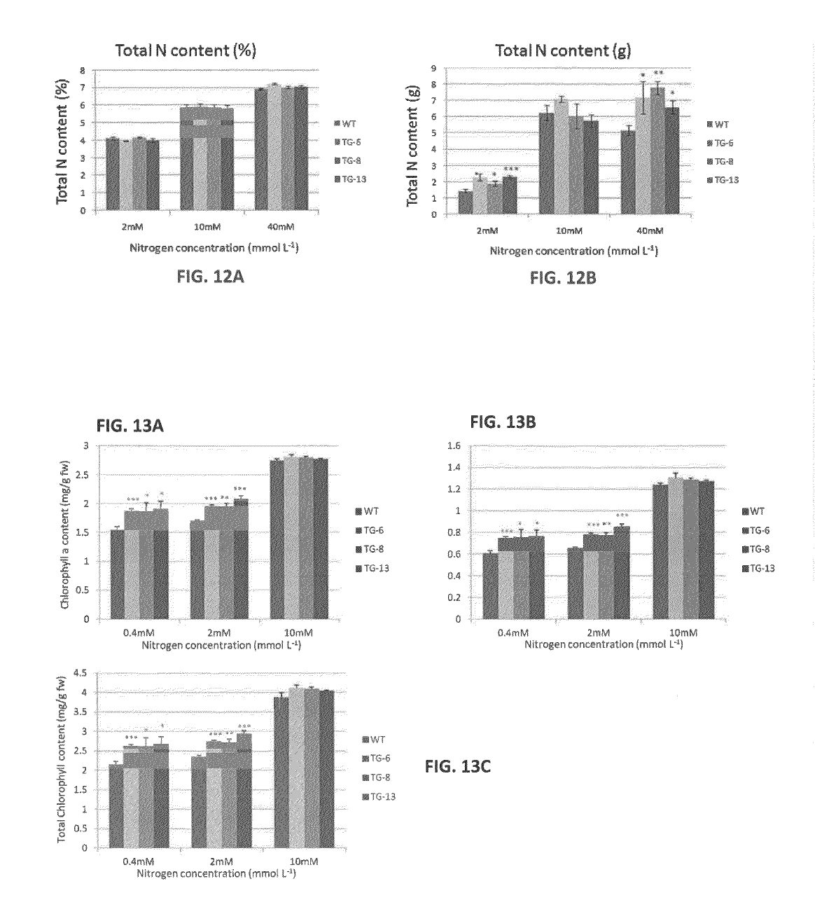

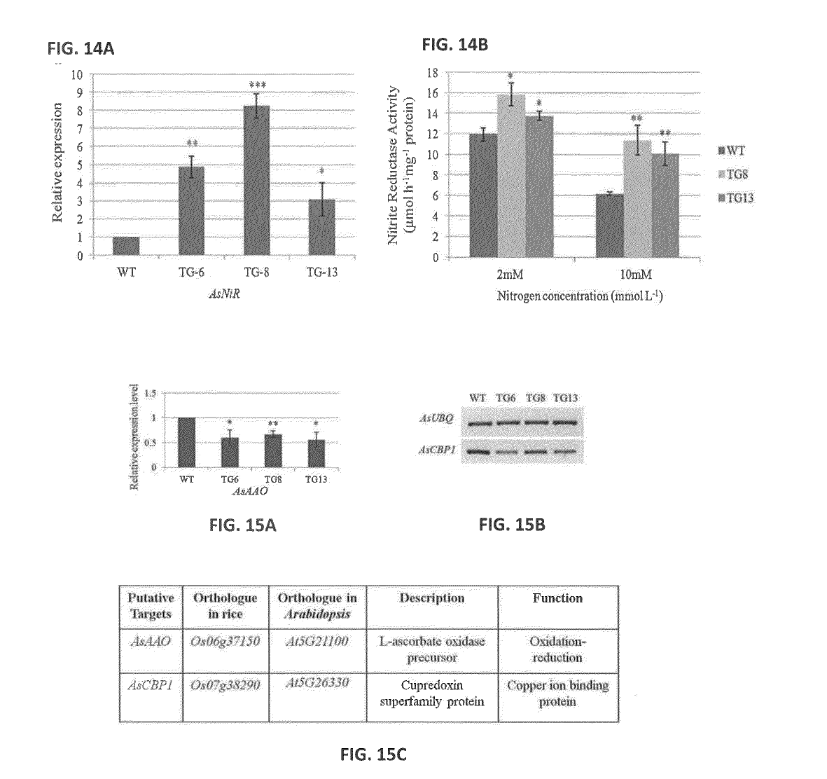

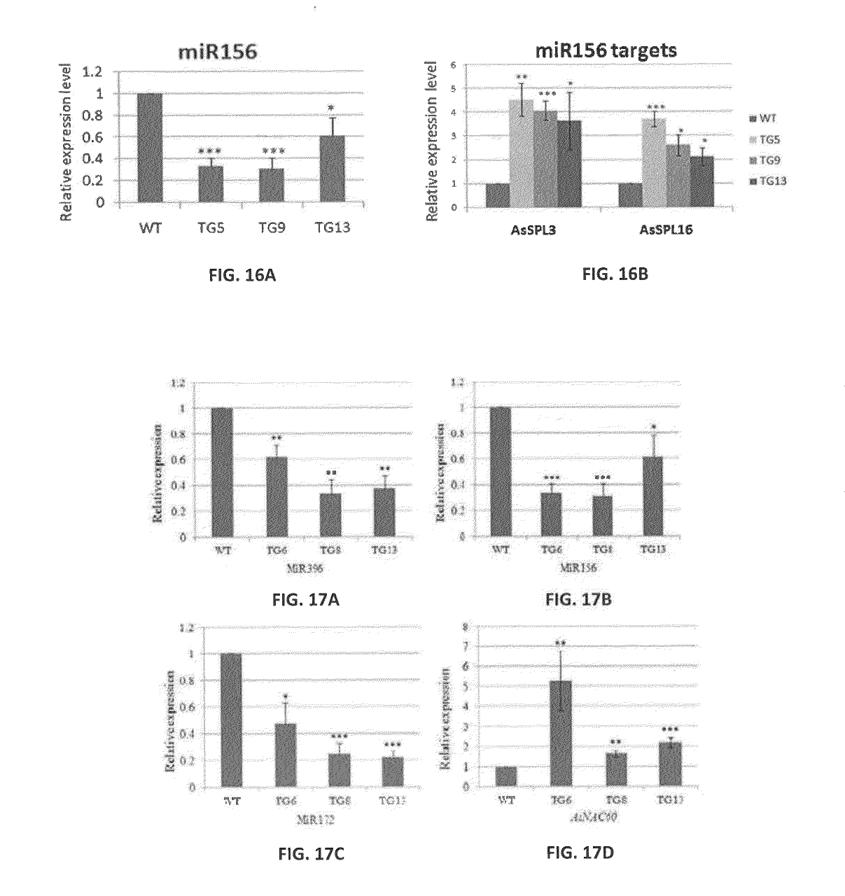

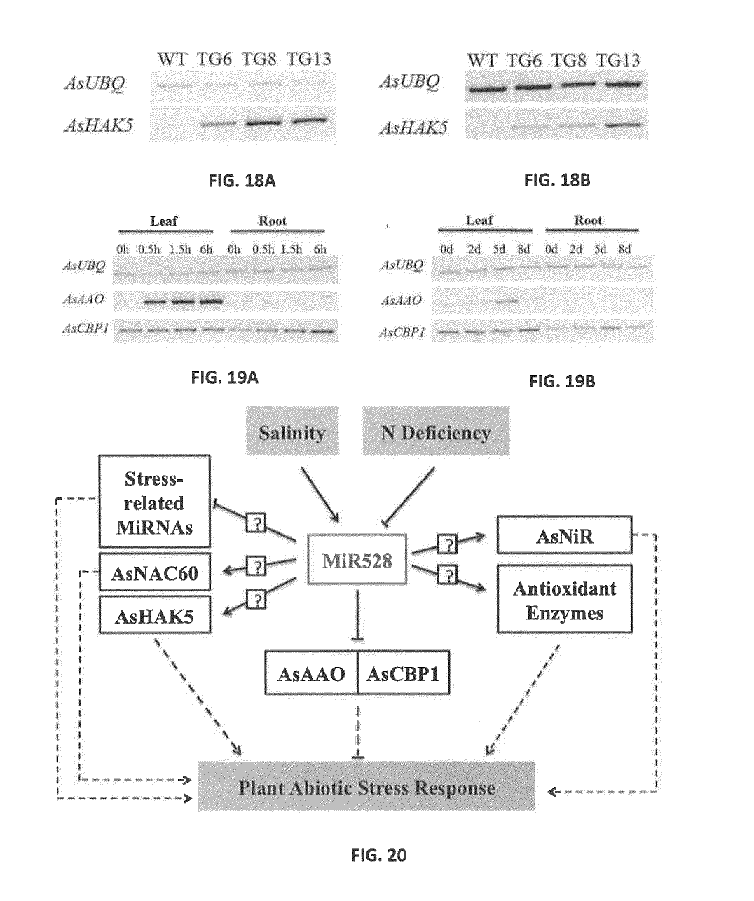

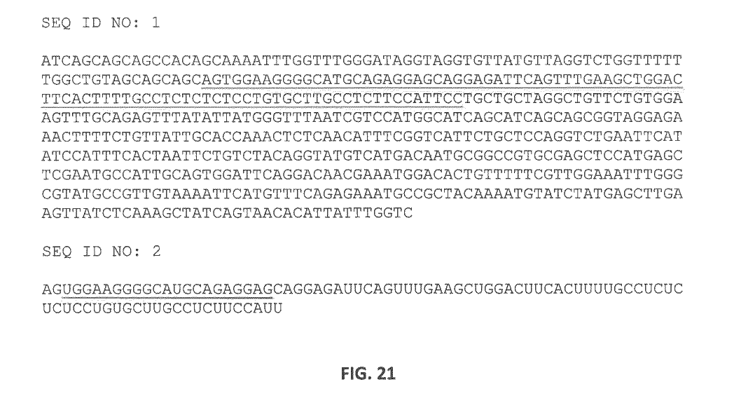

FIG. 4C illustrates the average length of top eight internodes from WT and TG tillers (n=6).

FIG. 5A illustrates shoot fresh weight n WT and TG plants 10 weeks after initiation from a single tiller (n=4).

FIG. 5B illustrates shoot dry weight of WT and TG plants 10 weeks after initiation from a single tiller (n=4).

FIG. 5C illustrates root fresh weight of WT and TG plants 10 weeks after initiation from a single tiller (n=4).

FIG. 5D presents root dry weight of WT and TG plants 10 weeks after initiation from a single tiller (n=4).

FIG. 5E presents biomass data from fully developed WT and TG plants grown in small Cone-tainers™ that were mowed weekly with the same height (n=4).

FIG. 5F illustrates the dry weight in WT and TG plants that were measured every week (n=4).

FIG. 6A presents images of WT controls and two TG lines that were trimmed to the same height before salt stress test.

FIG. 6B presents fully developed WT and TG plants initiating from the same amount of tillers that were subject to 200 mM salinity stress test.

FIG. 6C is a close up of representative WT and TG plants from FIG. 6B.

FIG. 6D graphically presents the electrolyte leakage values that were calculated at 9-day after salt stress treatment.

FIG. 6E presents the relative water contents as were measured 9-day after salt stress treatment. Data are presented as average (n=5), and error bars represent ±SE. Asterisks (*, or **) indicates a significant difference of EL or RWC between WT and transgenic plants at P<0.05 or 0.01 by Student's t-test.

FIG. 7A presents chlorophyll a contents of WT and TG under salt stress treatment, WT and TG leaves were collected before and 14-day after 200 mM NaCl treatment.

FIG. 7B presents chlorophyll b content of WT and TG under salt stress treatment. WT and TG leaves were collected before and 14-day after 200 mM NaCl treatment.

FIG. 7C presents total chlorophyll content of WT and TG under salt stress treatment. WT and TG leaves were collected before and 14-day after 200 mM NaCl treatment.

FIG. 8 presents the proline contents of WT and TG. WT and TG leaves were collected before and 14-day after 200 mM NaCl treatment. Proline content was measured. Data are presented as average (n=3), and error bars represent ±SE. Asterisks (*or **) indicates a significant difference of proline contents between WT and each transgenic lines at P<0.05, or 0.01 by Student's t-test.

FIG. 9A presents Na⁺ relative contents in shoot and root tissues of WT and TG plants before salinity treatment.

FIG. 9B presents Na⁺ relative contents in shoot and root tissues of WT and TG plants 9 days after salinity treatment.

FIG. 9C presents K⁺ relative contents in shoot and root tissues of \NT and TG plants under normal growth conditions.

FIG. 9D presents K⁺ relative contents in shoot and root tissues of WT and TG plants 9 days after salinity treatment.

FIG. 9E presents K⁺:Na⁺ ratio in shoots and roots of WT and TG plants before 200 mM NaCl treatment.

FIG. 9F presents K⁺:Na⁺ ratio in shoots and roots of WT and TG plants 9 days after salt treatment.

FIG. 9G presents shoot K⁺ relative contents in WT and TG plants before and after salinity stress.

FIG. 9H presents root K⁺ relative contents in WT and TG plants before and after salinity stress.

FIG. 10A presents catalase (CAT) activity measurement under normal and salt stress conditions.

FIG. 10B presents ascorbic acid oxidase (AAO) activity measurement under normal and salt stress conditions.

FIG. 11A illustrates WT and TG plants trimmed to be uniform before applying nitrogen solutions.

FIG. 11B illustrates the performance of WT controls and three TG lines applied with 2 mM, 10 mM, and 40 mM nitrate MS solutions for four weeks.

FIG. 11C illustrates close up views of WT and TG shoots under 2 mM nitrate MS solution treatment for four weeks.

FIG. 11D illustrates close up views of WT and TG shoots under 40 mM nitrate MS solution treatment for four weeks.

FIG. 11E presents the shoot fresh weight of WT and TG plants after 4-week growth with three different nitrate solution.

FIG. 11F presents the shoot dry weight of WT and TG plants after 4-week growth with three different nitrate solution.

FIG. 12A presents the total Nitrogen Content in WT & TG under different nitrogen concentrations as a percentage

FIG. 12B presents the total Nitrogen Content in WT & TG under different nitrogen concentrations by total nitrogen content.

FIG. 13A presents chlorophyll a content of WT and TG under different nitrogen concentrations. WT and TG leaves were collected four weeks after subjected to different concentrations of nitrogen supply.

FIG. 13B presents chlorophyll b content of WT and TG under different nitrogen concentrations. WT and TG leaves were collected four weeks after subjected to different concentrations of nitrogen supply.

FIG. 13C presents total chlorophyll content of WT and TG under different nitrogen concentrations. WT and TG leaves were collected four weeks after subjected to different concentrations of nitrogen supply.

FIG. 14A presents RT-qPCR analysis of AsNiR transcript levels in WT plants and three transgenic lines. AsACT1 was used as an endogenous control. Data are presented as means of three technical replicates and three biological replicates.

FIG. 14B presents NiR assay in WT controls and two transgenic lines before and two weeks after N starvation. Data are presented as means of three biological replicates.

FIG. 15A presents the expression levels of AsAAO in WT and three transgenic lines examined via RT-qPCR. Three biological replicates each having three technical replicates were used for analysis.

FIG. 15B presents semi-quantitative RT-PCR analysis of AsCBP1 expression in WT and TG plants. AsUBQ5 was used as an endogenous control.

FIG. 15C presents Information about the orthologues of the two putative miR528 target genes in rice and Arabidopsis.

FIG. 16A presents real-time PCR analysis of miR156 and its targets (FIG. 16B) AsSPL3, AsSPL16 expression level in WT and TG plants.

FIG. 17A presents expression levels of (a) miR396, ire WT and TG plants revealed through stem-loop RT-qPCR analysis.

FIG. 17B presents expression level of (b) miR156, and (c) in WT and TG plants revealed through stem-loop RT-qPCR analysis.

FIG. 17C presents expression level of miR172 in WT and TG plants revealed through stem-loop RT-qPCR analysis. (d)

FIG. 17D presents expression levels of AsNAC60 in WT and three transgenic lines through RT-qPCR analysis.

FIG. 18A presents expression levels of AsHAK5 in leaf tissues of WT and TG plants under normal growth conditions. AsUBQ5 gene was used as the endogenous control.

FIG. 18B presents expression levels of AsHAK5 in root tissues of WT and TG plants under normal growth conditions. AsUBQ5 gene was used as the endogenous control.

FIG. 19A presents expression profiles of the AsAAO and AsCBP1 in WT leaf and root tissues under 200 mM NaCl treatment (0 to 6 hours). AsUBQ5 gene was used as the endogenous control.

FIG. 19B presents expression profiles of the AsAAO and AsCBP1 in WT leaf and root tissues under N starvation (0 mM N) from 0 to 8 days. AsUBQ5 was used as an endogenous control.

FIG. 20 presents hypothetical model of molecular mechanisms of miR528-mediated plant abiotic stress response in creeping bentgrass.

FIG. 21 presents the cDNA sequence of the miR528 gene (SEQ ID NO: 1) with the sequence corresponding to the stem/loop sequence underlined and the stem/loop sequence of miR528 (SEQ ID NO: 2) with the miRNA sequence underlined.

### DETAILED DESCRIPTION

Reference will now be made in detail to various embodiments of the disclosure, one or more examples of which are illustrated in the accompanying drawings. Each example is provided by way of explanation of the subject matter, not limitation thereof. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

In general, the present disclosure is directed to a conserved monocot-specific miRNA, miR528 that can be utilized for mediating multiple stress responses and/or mediating morphological aspects of plant development. For instance, in one embodiment the present disclosure is directed to transgenic plant cells that have been transformed to include a recombinant nucleotide that encodes miR528. In one embodiment, the recombinant nucleotide can include the cDNA sequence of the miR528 gene, SEQ ID NO: 1 (FIG. 21, with the sequence corresponding to the stem/loop sequence underlined) in operative association with a promoter, or the stem/loop sequence of miR528, SEQ ID NO: 2 (FIG. 21, with the miRNA sequence underlined) in operative association with a promoter. The present disclosure is also directed to plant parts such as seeds and plants developed from the transgenic cells as well as progeny of the seeds and plants. Also disclosed are targets of miR528, all of which appear to function in oxidation-reduction processes.

Without wishing to be bound to any particular theory, it is believed that both plant development and stress response can be altered in miR528 transgenic plants. Morphologically, the miR528 transgenic plants can display shorter internodes, more tillers and more upright growth than wild-type (WT) controls (i.e., a naturally occurring or endogenous plant that is not transformed with miR528). Resistance to abiotic stresses and in one particular embodiment salt stress and/or nitrogen deficiency can also be enhanced in the transgenics. Improved salt stress resistance can be associated with one or more of increased water retention, cell membrane integrity, and chlorophyll content, while enhanced tolerance to nitrogen deficiency can be associated with one or more of increased biomass, total nitrogen and chlorophyll content.

Also disclosed are direct and indirect target genes of miR528. Direct target genes are those to which the miRNA is believe to directly interact with and encourage translational repression, mRNA degradation, or the like. Direct target genes can include, without limitation, AsAAO (SEQ ID NO: 3 (an AsAAO orthologue in A. stolonifera partial mRNA sequence), SEQ ID NO: 4 (an AsAAO orthologue in rice cDNA sequence), and SEQ ID NO: 5 (an AsAAO orthologue in Arabidopsis mRNA sequence)) and AsCSD1 (SEQ ID NO: 6 (an AsCSD1 orthologue in A. stolonifera partial mRNA sequence), SEQ ID NO: 7 (an AsCSD1 orthologue in rice mRNA sequence) and SEQ ID NO: 8 (an AsCSD1 orthologue in Arabidopsis mRNA sequence)), which function in oxidation-reduction. In one embodiment, disclosed is a recombinant nucleotide sequence that includes a polynucleotide that is antisense to only a portion of consecutive nucleotides (for instance more than about 20 but not all) of the sequence of SEQ ID NO: 3, SEQ ID NO: 4, SEQ ID NO: 5, SEQ ID NO: 6, SEQ ID NO: 7, or SEQ ID NO: 8. Also disclosed is a recombinant nucleotide including a nucleotide sequence that encodes only a portion of consecutive nucleotides of SEQ ID NO: 3, SEQ ID NO: 4, SEQ ID NO: 5, SEQ ID NO: 6, SEQ ID NO: 7, or SEQ ID NO: 8, which when expressed produces an antisense nucleotide sequence, wherein a plant expressing the antisense nucleotide sequence exhibits increased tolerance to abiotic stress as compared to a plant lacking the recombinant nucleotide.

As utilized herein, the term “recombinant polynucleotide” refers to a non-natural polynucleotide that has been altered, rearranged or modified from the natural state of the polynucleotide. For instance, the polynucleotide may be cloned or linked/joined to a heterologous sequence to which it is not naturally linked or joined.

Indirect target genes include those for which the expression of miR528 appears to have an effect, but this effect does not appear to be direct binding with the gene. Indirect targets can include AsNir, which encodes for nitrite reductase. Specifically, reductase level can be increased in transgenic plants as compared to WT controls, which is believed to contribute to enhanced nitrogen use efficiency. Other indirect target genes of miR528 are believed to be, without limitation, AsSPL3, AsSPL11, AsSPL16 AsNAC60, AsDREB2B, AsCSD2 (SEQ ID NO: 9, SEQ ID NO: 10).

A recombinant polynucleotide can include a nucleotide sequence as disclosed herein operatively linked to a heterologous nucleotide sequence. For instance, the heterologous nucleotide sequence can be one that is not present in conjunction with the miR528 nucleotide sequence in a naturally occurring plant. For example, the recombinant polynucleotide can include nucleotide sequence operatively linked to a heterologous promoter. The heterologous promoter can provide a means to express miR528 constitutively, inducibly, or in a tissue-specific or phase-specific manner.

As would be understood by those of skill in the art, any portion of a nucleotide sequence encoding miR528 that can function as microRNA is encompassed herein. Accordingly, any portion of an miR528 nucleotide sequence that comprises the stem-loop structure of the miR528 (e.g., an miR528 nucleotide sequence of SEQ ID NO: 1, and/or the nucleotide sequence of SEQ ID NO: 2, and/or any combination thereof) can be used to prepare the recombinant nucleic acid molecules. As known in the art, a processed miRNA transcript can be from about 19 to about 24 nucleotides in length. Therefore, in some embodiments of the invention, the processed miR528 can be about 19 to about 24 nucleotides in length.

One aspect of the present disclosure provides a recombinant nucleotide comprising a polynucleotide that hybridizes to the complement of a polynucleotide that encodes miR528 that can function as an miRNA, e.g., SEQ ID NO: 1 or SEQ ID NO: 2, which is operably linked to a regulatory element or functional portion thereof.

Hybridization conditions can be, for example:

(1) 7% sodium dodecyl sulfate (SDS), 0.5 M NaPO₄, 1 mM ethylenediamine tetraacetic acid (EDTA) at 50° C. with a final wash in 2× standard saline citrate (SSC), 0.1% SDS at 50° C.;

(ii) 7% SDS, 0.5 M NaPO₄, 1 mM EDTA at 50° C. with a final wash in 1×SSC, 0.1% SDS at 50° C.;

(iii) 7% SDS, 0.5 M NaPO₄, 1 mM EDTA at 50° C. with a final wash in 0.5×SSC, 0.1% SDS at 50° C.;

(iv) 7% SOS, 0.5 M NaPO₄, 1 mM EDTA at 50° C. with a final wash in 0.1×SSC, 0.1% SOS at 50° C.; and

(v) 7% SOS, 0.5 M NaPO₄, 1 mM EDTA at 50° C. with a final wash in 0.1×SSC, 0.1% SDS at 65° C.

When hybridization is performed under stringent conditions, the nucleic acid molecule can be present on a support; e.g., on a membrane or on a DNA chip. For instance, either a denatured test or nucleic acid molecule of the presently disclosed subject matter is first bound to a support and hybridization is effected for a specified period of time under conditions as described above.

One specific embodiment is directed to a recombinant nucleic acid molecule comprising a polynucleotide selected from the group consisting of: a) SEQ ID NO: 1; b) SEQ ID NO: 2; c) a polynucleotide that is antisense to only a portion of consecutive nucleotides of the sequence of any one or more of SEQ ID NO: 3-8; d) a nucleotide sequence that encodes only a portion of consecutive nucleotides of any one or more of SEQ ID NO: 3-8, which when expressed produces an antisense nucleotide sequence; d) a sequence that hybridizes under any of the hybridization conditions (i), (ii), (iii), (iv) or (v) to a polynucleotide of a), b), c), or d); e) the complement of any sequence of a), b), c) d); or e); f) the reverse complement of any sequence of a), b), c), d), or e); and g) an allelic variant of any of the above.

Also provided are expression cassettes, plants, and seeds comprising any of the disclosed isolated sequences.

According to another embodiment, disclosed is a method of producing a transgenic plant that includes at least one plant cell that exhibits altered responsiveness to a stress condition, particularly an abiotic stress, and more particularly water and/or nitrogen stress. In one embodiment, the method can be performed by introducing a nucleotide sequence comprising SEQ ID NO: 1 or SEQ ID NO: 2 operatively linked to a heterologous promoter into a plant cell genome, whereby the nucleotide sequence modulates a response of the plant cell to a stress condition. The nucleotide sequence can integrate into the plant cell genome in a site-specific manner, whereupon it can be operatively linked to a heterologous nucleotide sequence, which can be expressed in response to a stress condition specific for the regulatory element; or can be a mutant regulatory element, which is not responsive to the stress condition, whereby upon integrating into the plant cell genome, the mutant regulatory element disrupts an endogenous stress-regulated regulatory element of a plant stress-regulated nucleotide sequence, thereby altering the responsiveness of the plant stress-regulated nucleotide sequence to the stress condition.

According to another embodiment, disclosed are methods for down regulating expression of AsAO or AsCD1 or a functional equivalent thereof. In one embodiment, the method can be performed by introducing a coding sequence into a plant genome, for instance via an expression cassette. The coding region of the expression cassette can include a sequence encoding miR528.

Further aspects include plants and uniform populations of plants made by the above methods as well as seeds and progeny from such plants and cDNA or genomic DNA libraries prepared from the transgenic plant, or from a plant cell from said transgenic plant, wherein said plant cell exhibits altered responsiveness to the stress condition.

Transgenic plant cells, transgenic plants, and/or transgenic plant parts comprising a recombinant nucleic acid as described herein (e.g., a transgenic plant including a recombinant nucleic acid that comprises a nucleotide sequence encoding miR528) as well as crops comprising a plurality of the transgenic plants and methods of producing such plants are encompassed herein. Crops can include, for example, an agricultural field, a golf course, a residential lawn, a road side, an athletic field, and/or a recreational field.

The term “plant” means any plant and thus can include, without limitation, angiosperms, gymnosperms, bryophytes, ferns and/or fern allies. Non-limiting examples of plants can include turf grasses, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussel sprouts, cabbage, cauliflower, broccoli, collards, kale, Chinese cabbage, bok choy), cardoni, carrots, napa cabbage, okra, onions, celery, parsley, chick peas, parsnips, chicory, peppers, potatoes, cucurbits (e.g., marrow, cucumber, zucchini, squash, pumpkin), radishes, dry bulb onions, rutabaga, eggplant, salsify, escarole, shallots, endive, garlic, spinach, green onions, squash, greens, beet (sugar beet and fodder beet), sweet potatoes, swiss chard, horseradish, tomatoes, turnips, and spices; a fruit and/or vine crop such as apples, apricots, cherries, nectarines, peaches, pears, plums, prunes, cherry, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee, a field crop plant such as clover, alfalfa, evening primrose, meadow foam, corn/maize (field, sweet, popcorn), hops, jojoba, peanuts, rice, safflower, small grains (barley, oats, rye, wheat, etc.), sorghum, tobacco, kapok, a leguminous plant (beans, lentils, peas, soybeans), an oil plant (rape, mustard, poppy, olive, sunflower, coconut, castor oil plant, cocoa bean, groundnut), Arabidopsis, a fiber plant (cotton, flax, hemp, jute), lauraceae (cinnamon, camphor), or a plant such as coffee, sugar cane, tea, and natural rubber plants; and/or a bedding plant such as a flowering plant, a cactus, a succulent and/or an ornamental plant, as well as trees such as forest (broad-leaved trees and evergreens, such as conifers), fruit, ornamental, and nut-bearing trees, as well as shrubs and other nursery stock.

In particular embodiments, a plant cell and/or plant is a turfgrass. Turfgrass can include, but is not limited to, Sporobolus airiodes, Puccinellia distans, Paspalum notatum, Cynodon dactylon, Buchloe dactyloides, Cenchrus cillaris, Hordeum califormicum, Hordeum vulgare, Hordeum brachyantherum, Agrostis capillaries, Agrostis palustris, Agrostis exerata, Brize maxima, Poa annua, Poe ampla, Poe canbyi, Poe compressa, Poa pratensis, Poa scabrella, Poe trivialis, Poe secunda, Andropogon gerardii, Schizachyruim scoparium, Andropogon hallii, Bromus arizonicus, Bromus carinatus, Bromus biebersteinii, Bromus marginatus, Bromus rubens, Bromus inermis, Buchloe dactyloides, Axonopus fussifolius, Eremochloa ophiuroides, Muhlenbergia rigens, Sporobolus cryptandrus, Sporobolus heterolepis, Tripsacum dactyloides, Festuca arizonica, Festuca rubra var. commutate, Festuca rubra var. rubra, Festuca megalura, Festuca longifolia, Festuca idahoensis, Festuca elation, Fescue rubra, Fescue ovine var. ovina, Festuca arundinacea, Alopecurus arundinaceaus, Alopecurus pratensis, Hilaria jamesii, Bouteloua eriopoda, Bouteloua gracilis, Bouteloua curtipendula, Deschampsia caespitosa, Oryzopsis hymenoides, Sorghastrum nutans, Eragrostis trichodes, Eragrostis curvula, Melica californica, Stipa comate, Stipa lepida, Stipa viridula, Stipa cernua, Stipa pulchra, Dactylis glomerata, Koeleria pyramidata, Calamovilfa longifolia, Agrostis alba, Phalaris arundinacea, Stenotaphrum secundatum, Spartina pectinate, Lolium multiflorum, Lolium perenne, Leptochloa dubia, Sitanion hystrix, Panicum virgatum, Aristida purpurea, Phleum pretense, Agropyron spicatum, Agropyron cristatum, Agropyron desertorum, Agropyron intermedium, Agropyron trichophorum, Agropyron trachycaulum, Agropyron riparium, Agropyron elongatum, Agropyron smithii, Elymus glaucus, Elymus Canadensis, Elymus triticoides, Elymus junceus, Zoysia japonica, Zoysia matrella, and Zoysia tenuifolia. In some embodiments, a plant of the present invention is creeping bent grass, Agrostis palustris.

According to one embodiment, a method comprises introducing into a plant cell an expression cassette comprising a nucleic acid molecule of the presently disclosed subject matter as disclosed above to obtain a transformed plant cell or tissue, and culturing the transformed plant cell or tissue. The nucleic acid molecule can be under the regulation of a constitutive or inducible promoter. The method can further comprise inducing or repressing expression of a nucleic acid molecule that is directly or indirectly targeted by the miRNA in the plant for a time sufficient to modify (e.g., downregulate) the concentration and/or composition of the targeted expression product in the plant or plant part.

A plant or plant part transformed to include a recombinant nucleic acid molecule of the presently disclosed subject matter can be analyzed and selected using methods known to those skilled in the art including, but not limited to, Southern blotting, DNA sequencing, or PCR analysis using primers specific to the nucleic acid molecule and detecting amplicons produced therefrom.

In general, a concentration of an expression product of a gene targeted by miR528 can be decreased by at least in one embodiment 2%, in another embodiment 3%, in another embodiment 5%, in another embodiment 10%, in another embodiment 20%, in another embodiment 30%, in another embodiment 40%, in another embodiment 50%, relative to a native control plant, plant part, or cell lacking the recombinant nucleic acid molecule.

Transforming a cell with a nucleic acid molecule encoding miR528 can be accomplished using standard methods. For example, constitutive, inducible, tissue-specific, cell type-specific, or developmentally-regulated expression are within the scope of the presently disclosed subject matter and result in a constitutive, inducible, tissue-specific, or developmentally-regulated expression of miR528 in the plant cell.

Further encompassed within the presently disclosed subject matter is a recombinant vector comprising an expression cassette according to the embodiments of the presently disclosed subject matter. Also encompassed are plant cells comprising expression cassettes according to the present disclosure, and plants comprising these plant cells.

In one embodiment, the expression cassette is expressed throughout the plant. In another embodiment, the expression cassette is expressed in a specific location or tissue of a plant. In one embodiment, the location or tissue includes, but is not limited to, epidermis, root, vascular tissue, meristem, cambium, cortex, pith, leaf, flower, and combinations thereof. In another embodiment, the location or tissue is a seed.

In one embodiment, the expression cassette is involved in a function including, but not limited to, disease resistance, yield, biotic or abiotic stress resistance, nutritional quality, carbon metabolism, photosynthesis, signal transduction, cell growth, reproduction, disease processes (for example, pathogen resistance), gene regulation, and differentiation.

For example, a nucleic acid molecule encoding miR528 can be introduced, under conditions for expression, into a host cell such that the host cell transcribes and translates the nucleic acid molecule to produce the miRNA. By “under conditions for expression” is meant that a nucleic acid molecule is positioned in the cell such that it will be expressed in that cell. For example, a nucleic acid molecule can be located downstream of a promoter that is active in the cell, such that the promoter will drive the expression of the polypeptide encoded for by the nucleic acid molecule in the cell. Any regulatory sequence (e.g., promoter, enhancer, inducible promoter) can be linked to the nucleic acid molecule; alternatively, the nucleic acid molecule can include its own regulatory sequence(s) such that it will be expressed (i.e., transcribed and/or translated) in a cell.

Where the nucleic acid molecule is introduced into a cell under conditions of expression, that nucleic acid molecule can be included in an expression cassette. Thus, the presently disclosed subject matter further provides a host cell comprising an expression cassette comprising a nucleic acid molecule encoding an miR528. Such an expression cassette can include, in addition to the nucleic acid molecule encoding miR528, at least one regulatory sequence (e.g., a promoter and/or an enhancer).

As such, coding sequences intended for expression in transgenic plants can be first assembled in expression cassettes operatively linked to a suitable promoter expressible in plants. The expression cassettes can also comprise any further sequences required or selected for the expression of the transgene. Such sequences include, but are not limited to, transcription terminators, extraneous sequences to enhance expression such as introns, vital sequences, and sequences intended for the targeting of the gene product to specific organelles and cell compartments. These expression cassettes can then be easily transferred to the plant transformation vectors disclosed below. The following is a description of various components of typical expression cassettes.

The selection of the promoter used in expression cassettes can determine the spatial and temporal expression pattern of the miR528 in the transgenic plant. Selected promoters can express transgenes in specific cell types (such as leaf epidermal cells, mesophyll cells, root cortex cells) or in specific tissues or organs (roots, leaves, or flowers, for example) and the selection can reflect the desired location for accumulation of the gene product. Alternatively, the selected promoter can drive expression of the gene under various inducing conditions. Promoters vary in their strength; i.e., their abilities to promote transcription. Depending upon the host cell system utilized, any one of a number of suitable promoters can be used, including the gene's native promoter. The following are non-limiting examples of promoters that can be used in expression cassettes.

In one non-limiting example, a plant promoter fragment can be employed that will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of ordinary skill in the art. Such genes include for example, the AP2 gene, ACT11 from Arabidopsis (Huang et al., 1996), Cat3 from Arabidopsis (GENBANK® Accession No. U43147; Zhong et al., 1996), the gene encoding stearoyl-acyl carrier protein desaturase from Brassica napus (GENBANK® Accession No. X74782; Solocombe et al., 1994), GPc1 from maize (GENBANK® Accession No. X15596; Martinez et al., 1989), and Gpc2 from maize (GENBANK® Accession No. U45855; Manjunath et al., 1997).

Alternatively, the plant promoter can direct expression of the nucleic acid molecules in a specific tissue or can be otherwise under more precise environmental or developmental control. Examples of environmental conditions that can effect transcription by inducible promoters include anaerobic conditions, elevated temperature, or the presence of light. Such promoters are referred to herein as “inducible”, “cell type-specific”, or “tissue-specific” promoters. Ordinary skill in the art will recognize that a tissue-specific promoter can drive expression of operatively linked sequences in tissues other than the target tissue. Thus, as used herein a tissue-specific promoter is one that drives expression preferentially in the target tissue, but can also lead to some expression in other tissues as well.

Examples of promoters under developmental control include promoters that initiate transcription only (preferentially) in certain tissues, such as fruit, seeds, or flowers. Promoters that direct expression of nucleic acids in ovules, flowers, or seeds are particularly useful in the presently disclosed subject matter. As used herein a seed-specific or preferential promoter is one that directs expression specifically or preferentially in seed tissues. Such promoters can be, for example, ovule-specific, embryo-specific, endosperm-specific, integument-specific, seed coat-specific, or some combination thereof, Examples include a promoter from the ovule-specific BEL1 gene described in Reiser et al., 1995 (GENBANK® Accession No. U39944), Non-limiting examples of seed specific promoters are derived from the following genes: MAC1 from maize (Sheridan et al., 1996), Cat3 from maize (GENBANK® Accession No. L05934; Abler et al., 1993), the gene encoding oleosin 18 kD from maize (GENBANK® Accession No. J05212; Lee et al., 1994), vivparous-1 from Arabidopsis (GENBANK® Accession No. U93215), the gene encoding oleosin from Arabidopsis (GENBANK® Accession No. Z17657), Atmycl from Arabidopsis (Urao et al., 1996), the 2s seed storage protein gene family from Arabidopsis (Conceicao et al., 1994) the gene encoding oleosin 20 kD from Brassica napus (GENBANK Accession No. M63985), napA from Brassica napus (GENBANK® Accession No. J02798; Josefsson et al., 1987), the napin gene family from Brassica napus (Sjodahl et al., 1995), the gene encoding the 2S storage protein from Brassica napus (Dasgupta et al., 1993), the genes encoding oleosin A (GENBANK® Accession No. U09118) and oleosin B (GENBANK® Accession No. U09119) from soybean, and the gene encoding low molecular weight sulphur rich protein from soybean (Choi et al., 1995).

Alternatively, particular sequences that provide the promoter with desirable expression characteristics, or the promoter with expression enhancement activity, could be identified and these or similar sequences introduced into the sequences via cloning or via mutation. It is further contemplated that these sequences can be mutagenized in order to enhance the expression of transgenes in a particular species.

Furthermore, it is contemplated that promoters combining elements from more than one promoter can be employed. For example, U.S. Pat. No. 5,491,288 (incorporated herein by reference) discloses combining a Cauliflower Mosaic Virus (CaMV) promoter with a histone promoter. Thus, the elements from the promoters disclosed herein can be combined with elements from other promoters.

Another pattern of gene expression is root expression. A suitable root promoter is the promoter of the maize metallothionein-like (MTL) gene disclosed in de Framond, 1991, and also in U.S. Pat. No. 5,466,785, each of which is incorporated herein by reference. This “MTL” promoter is transferred to a suitable vector such as pCGN1761 ENX for the insertion of a selected gene and subsequent transfer of the entire promoter-gene-terminator cassette to a transformation vector of interest.

Wound-inducible promoters can also be suitable for gene expression. Numerous such promoters have been disclosed (e.g., Xu et al., 1993; Logemann et al., 1989; Rohrmeier & Lehle, 1993; Firek et al., 1993; Warner et al., 1993) and all are suitable for use with the presently disclosed subject matter. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA that is wound induced and which can be used to isolate the cognate promoter using standard techniques. Similarly, Firek et al. and Warner et al. have disclosed a wound-induced gene from the monocotyledon Asparagus officinalis, which is expressed at local wound and pathogen invasion sites. Using cloning techniques well known in the art, these promoters can be transferred to suitable vectors, fused to the genes pertaining to the presently disclosed subject matter, and used to express these genes at the sites of plant wounding.

PCT International Publication WO 93/07278, which is herein incorporated by reference, describes the isolation of the maize trpA gene, which is preferentially expressed in pith cells. The gene sequence and promoter extending up to −1726 base pairs (bp) from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

A maize gene encoding phosphoenol carboxylase (PEPC) has been disclosed by Hudspeth & Grula, 1989. Using standard molecular biological techniques, the promoter for this gene can be used to drive the expression of any gene in a leaf-specific manner in transgenic plants.

WO 93/07278 (incorporated herein by reference) describes the isolation of the maize calcium-dependent protein kinase (CDPK) gene that is expressed in pollen cells. The gene sequence and promoter extend up to 1400 by from the start of transcription. Using standard molecular biological techniques, this promoter or parts thereof can be transferred to a vector such as pCGN1761 where it can replace the 35S promoter and be used to drive the expression of a nucleic acid sequence of the presently disclosed subject matter in a pollen-specific manner.

A variety of 5 and 3′ transcriptional regulatory sequences are available for use in the presently disclosed subject matter. Transcriptional terminators are responsible for the termination of transcription and correct mRNA polyadenylation. The 3′ nontranslated regulatory DNA sequence includes from in one embodiment about 50 to about 1,000, and in another embodiment about 100 to about 1,000, nucleotide base pairs and contains plant transcriptional and translational termination sequences. Appropriate transcriptional terminators and those that are known to function in plants include the CaMV 353 terminator, the tml terminator, the nopaline synthase terminator, the pea rbcS E9 terminator, the terminator for the T7 transcript from the octopine synthase gene of Agrobacterium tumefaciens, and the 3 end of the protease inhibitor I or II genes from potato or tomato, although other 3′ elements known to those of skill in the art can also be employed. Alternatively, a gamma coixin, oleosin 3, or other terminator from the genus Coix can be used.

Non-limiting 3′ elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor I or II genes from potato or tomato.

As the DNA sequence between the transcription initiation site and the start of the coding sequence (i.e., the untranslated leader sequence, also referred to as the 5′ untranslated region) can influence gene expression, a particular leader sequence can also be employed. Non-limiting leader sequences are contemplated to include those that include sequences predicted to direct optimum expression of the operatively linked gene; i.e., to include a consensus leader sequence that can increase or maintain mRNA stability and prevent inappropriate initiation of translation. The choice of such sequences will be known to those of skill in the art in light of the present disclosure. Sequences that are derived from genes that are highly expressed in plants are useful in the presently disclosed subject matter.

Thus, a variety of transcriptional terminators are available for use in expression cassettes. These are responsible for termination of transcription and correct mRNA polyadenylation. Appropriate transcriptional terminators are those that are known to function in plants and include the CaMV 35S terminator, the tml terminator, the nopaline synthase terminator, and the pea rbcS E9 terminator. These can be used in both monocotyledons and dicotyledons. In addition, a gene's native transcription terminator can be used.

Numerous sequences have been found to enhance gene expression from within the transcriptional unit and these sequences can be used in conjunction with the miR528 sequences to increase theft expression in transgenic plants.

Other sequences that have been found to enhance gene expression in transgenic plants include intron sequences (e.g., from Adhl, bronze1, actin1, actin 2 (PCT International Publication No, WO 00/760067 (incorporated herein by reference)), or the sucrose synthase intron), and viral leader sequences (e.g., from Tobacco Mosaic Virus (TMV), Maize Chlorotic Mottle Virus (MCMV), or Alfalfa Mosaic Virus (AMV)). For example, a number of non-translated leader sequences derived from viruses are known to enhance the expression of operatively linked nucleic acids. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include, but are not limited to picornavirus leaders, for example, encephalomyocarditis virus (EMCV) leader (encephalomyocarditis 5′ noncoding region; Elroy-Stein et al., 1989); potyvirus leaders (e.g., Tobacco Etch Virus (TEV) leader and Maize Dwarf Mosaic Virus (MDMV) leader); human immunoglobulin heavy-chain binding protein (BIP) leader (Macejak et al., 1991); untranslated leader from the coat protein mRNA of AMV (AMV RNA 4; Jobling et al., 1987); TMV leader (Gallie et al., 1989); and maize chlorotic mottle virus leader (Lommel et al., 1991). See also, Della-Cioppa et al., 1987. Regulatory elements such as Adh intron 1 (Callis et al., 1987), sucrose synthase intron (Vasil et al., 1989) or TMV omega element (Gallie et al., 1989), can further be included where desired. Non-limiting examples of enhancers include elements from the CaMV 355 promoter, octopine synthase genes (Ellis et al., 1987), the rice actin I gene, the maize alcohol dehydrogenase gene (Callis et al., 1987), the maize shrunken I gene (Vasil et al., 1989), TMV omega element (Gallie et al., 1989) and promoters from non-plant eukaryotes (e.g., yeast; Ma et al., 1988).

A number of non-translated leader sequences derived from viruses are also known to enhance expression, and these are particularly effective in dicotyledonous cells, Specifically, leader sequences from Tobacco Mosaic Virus (TMV; the “W-sequence”), Maize Chlorotic Mottle Virus (MCMV), and Alfalfa Mosaic Virus (AMV) have been shown to be effective in enhancing expression (see e.g., Gallie et al., 1987; Skuzeski et al., 1990), Other leader sequences known in the art include, but are not limited to, picornavirus leaders, for example, EMCV (encephalomyocarditis virus) leader (5′ noncoding region; see Elroy-Stein et al., 1989); potyvirus leaders, for example, from Tobacco Etch Virus (TEV; see Allison et al., 1986); Maize Dwarf Mosaic Virus (MDMV; see Kong & Steinbiss 1998); human immunoglobulin heavy-chain binding polypeptide (BiP) leader (Macejak & Sarnow, 1991); untranslated leader from the coat polypeptide mRNA of alfalfa mosaic virus (AMV; RNA 4; see Jobling & Gehrke, 1987); tobacco mosaic virus (TMV) leader (Gallie et al., 1989); and Maize Chlorotic Mottle Virus (MCMV) leader (Lommel et al., 1991). See also, Della-Cioppa et al., 1987.

In addition to incorporating one or more of the aforementioned elements into the 5′ regulatory region of a target expression cassette of the presently disclosed subject matter, other elements can also be incorporated. Such elements include, but are not limited to, a minimal promoter. By minimal promoter it is intended that the basal promoter elements are inactive or nearly so in the absence of upstream or downstream activation. Such a promoter has low background activity in plants when there is no transactivator present or when enhancer or response element binding sites are absent. One minimal promoter that is particularly useful for target genes in plants is the Bz1 minimal promoter, which is obtained from the bronze1 gene of maize. The Bz1 core promoter is obtained from the “myc” mutant Bz1-luciferase construct pBz1LucR98 via cleavage at the Nhel site located at positions −53 to −58 (Roth et al., 1991). The derived Bz1 core promoter fragment thus extends from positions −53 to +227 and includes the Bz1 intron-1 in the 5′ untranslated region. Also useful for the presently disclosed subject matter is a minimal promoter created by use of a synthetic TATA element. The TATA element allows recognition of the promoter by RNA polymerase factors and confers a basal level of gene expression in the absence of activation (see generally, Mukumoto et al., 1993; Green, 2000.

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various polypeptides that is cleaved during chloroplast import to yield the mature polypeptides (see e.g., Comai et al., 1988), These signal sequences can be fused to heterologous gene products to affect the import of heterologous products into the chloroplast (Van den Broeck et al., 1985). DNA encoding for appropriate signal sequences can be isolated from the 5′ end of the cDNAs encoding the ribulose-1,5-bisphosphate carboxylase/oxygenase (RUBISCO) polypeptide, the chlorophyll a/b binding (CAB) polypeptide, the 5-enol-pyruvyl shikimate-3-phosphate (EPSP) synthase enzyme, the GS2 polypeptide and many other polypeptides which are known to be chloroplast localized. See also, the section entitled “Expression With Chloroplast Targeting” in Example 37 of U.S. Pat. No. 5,639,949, herein incorporated by reference,

Other gene products can be localized to other organelles such as the mitochondrion and the peroxisome (e.g., Unger et al., 1989). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular polypeptide bodies has been disclosed by Rogers et al., 1985.

In addition, sequences have been characterized that control the targeting of gene products to other cell compartments. Amino terminal sequences are responsible for targeting to the endoplasmic reticulum (ER), the apoplast, and extracellular secretion from aleurone cells (Koehler & Ho, 1990). Additionally, amino terminal sequences in conjunction with carboxy terminal sequences are responsible for vacuolar targeting of gene products (Shinshi et al., 1990).

By the fusion of the appropriate targeting sequences disclosed above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected can include the known cleavage site, and the fusion construct can take into account any amino acids after the cleavage site that are required for cleavage. In some cases this requirement can be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques disclosed by Bartlett et al., 1982 and Wasmann et al., 1986. These construction techniques are well known in the art and are equally applicable to mitochondria and peroxisomes.

The above-disclosed mechanisms for cellular targeting can be utilized not only in conjunction with their cognate promoters, but also in conjunction with heterologous promoters so as to effect a specific cell-targeting goal under the transcriptional regulation of a promoter that has an expression pattern different from that of the promoter from which the targeting signal derives.

Once an miR528 nucleic acid construct has been cloned into an expression system, it can be transformed into a plant cell. The receptor and target expression cassettes of the presently disclosed subject matter can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Transformation of a plant can be undertaken with a single DNA molecule or multiple DNA molecules (i.e., co-transformation), and both these techniques are suitable for use with the expression cassettes of the presently disclosed subject matter. Numerous transformation vectors are available for plant transformation, and the expression cassettes of the presently disclosed subject matter can be used in conjunction with any such vectors. The selection of vector will depend upon the transformation technique and the species targeted for transformation.

A variety of techniques are available and known for introduction of nucleic acid molecules and expression cassettes comprising such nucleic acid molecules into a plant cell host. These techniques include, but are not limited to transformation with DNA employing A. tumefaciens or A. rhizogenes as the transforming agent, liposomes, PEG precipitation, electroporation, DNA injection, direct DNA uptake, microprojectile bombardment, particle acceleration, and the like (see e.g., EP 0 295 959 and EP 0 138 341).

Expression vectors containing genomic or synthetic fragments can be introduced into protoplasts or into intact tissues or isolated cells. In some embodiments, expression vectors are introduced into intact tissue. “Plant tissue” includes differentiated and undifferentiated tissues or entire plants, including but not limited to roots, stems, shoots, leaves, pollen, seeds, tumor tissue, and various forms of cells and cultures such as single cells, protoplasts, embryos, and callus tissues. The plant tissue can be in plants or in organ, tissue, or cell culture. General methods of culturing plant tissues are provided, for example, by Maki et al., 1993 and by Phillips et al. 1988. In some embodiments, expression vectors are introduced using a direct gene transfer method such as microprojectile-mediated delivery, DNA injection, electroporation, or the like. In some embodiments, expression vectors are introduced into plant tissues using microprojectile media delivery with a biolistic device (see e.g., Tomes et al., 1995). The vectors can not only be used for expression of structural genes but can also be used in exon-trap cloning or in promoter trap procedures to detect differential gene expression in varieties of tissues (Lindsey et al., 1993; Auch & Reth, 1990).

In some embodiments, the binary type vectors of the Ti and Ri plasmids of Agrobacterium spp. are employed. Ti-derived vectors can be used to transform a wide variety of higher plants, including monocotyledonous and dicotyledonous plants including, but not limited to soybean, cotton, rape, tobacco, and rice (Pacciotti et al., 1985: Byrne et al., 1987; Sukhapinda et al., 1987; Lorz et al., 1985; Potrykus, 1985; Park et al., 1985: Hiel et al., 1994). The use of T-DNA to transform plant cells has received extensive study and is amply described (European Patent Application No. EP 0 120516; Hoekema, 1985; Knauf et al., 1983; and An et al., 1985, each of which is incorporated by reference in its entirety).

Other transformation methods are available to those skilled in the art, such as direct uptake of foreign DNA constructs (see European Patent Application No. EP 0 295 959), electroporation (Fromm et al., 1986), or high velocity ballistic bombardment of plant cells with metal particles coated with the nucleic acid constructs (Kline et al., 1987; U.S. Pat. No. 4,945,050). Once transformed, the cells can be regenerated using techniques familiar to those of skill hi the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1989; Christou et al., 1989; European Patent Application No. EP 0 301 749), rice (Hiei et al., 1994), and corn (Gordon Kamm et al., 1990; Fromm et al., 1990).

Of course, the choice of method might depend on the type of plant targeted for transformation. Suitable methods of transforming plant cells include, but are not limited to microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc. (Madison, Wis., United States of America) and BioRad (Hercules, Calif., United States of America). See e.g., U.S. Pat. No. 4,945,050; McCabe et al., 1988; Weissinger et al., 1988; Sanford et al., 1987 (onion); Christou et al., 1988 (soybean); McCabe et al., 1988 (soybean); Datta et al., 1990 (rice); Klein et al., 1988 (maize); Fromm et al., 1990 (maize); Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (see European Patent Application EP 0 292 435; U.S. Pat. No. 5,350,689).

Agrobacterium tumefaciens cells containing a vector comprising an expression cassette of the presently disclosed subject matter, wherein the vector comprises a Ti plasmid, are useful in methods of making transformed plants. Plant cells are infected with an Agrobacterium tumefaciens to produce a transformed plant cell, and then a plant is regenerated from the transformed plant cell. Numerous Agrobacterium vector systems useful in carrying out the presently disclosed subject matter are known to ordinary skill in the art.

Many vectors are available for transformation using Agrobacterium tumefaciens. These typically carry at least one T-DNA border sequence and include vectors such as pBIN19 (Bevan, 1984). For instance, the binary vectors pCIB200 and pCIB2001 can be used for the construction of recombinant vectors for use with Agrobacterium and can be constructed according to known methodology.

Transformation without the use of Agrobacterium tumefaciens circumvents the requirement for T-DNA sequences in the chosen transformation vector, and consequently vectors lacking these sequences can be utilized in addition to vectors such as the ones disclosed above that contain T-DNA sequences. Transformation techniques that do not rely on Agrobacterium include transformation via particle bombardment, protoplast uptake (e.g., polyethylene glycol (PEG) and electroporation), and microinjection. The choice of vector depends largely on the species being transformed.

Methods using either a form of direct gene transfer or Agrobacterium-mediated transfer usually, but not necessarily, are undertaken with a selectable marker that can provide resistance to an antibiotic (e.g., kanamycin, hygromycin, or methotrexate) or a herbicide (e.g., phosphinothricin). The choice of selectable marker for plant transformation is not, however, critical to the presently disclosed subject matter.

For certain plant species, different antibiotic or herbicide selection markers can be employed. Selection markers used routinely in transformation include the nptII gene, which confers resistance to kanamycin and related antibiotics (Messing & Vierra, 1982; Bevan et al., 1983), the bar gene, which confers resistance to the herbicide phosphinothricin (White et al., 1990, Spencer et al., 1990), the hph gene, which confers resistance to the antibiotic hygromycin (Blochinger & Diggelmann, 1984), and the dhfr gene, which confers resistance to methotrexate (Bourouis et al., 1983).

Selection markers resulting in positive selection, such as a phosphomannose isomerase (PMI) gene (described in PCT International Publication No. WO 93/05163) can also be used. Other genes that can be used for positive selection are described in PCT International Publication No. WO 94/20627 and encode xyloisomerases and phosphomanno-isomerases such as mannose-6-phosphate isomerase and mannose-1-phosphate isomerase; phosphomanno mutase; mannose epimerases such as those that convert carbohydrates to mannose or mannose to carbohydrates such as glucose or galactose; phosphatases such as mannose or xylose phosphatase, mannose-6-phosphatase and mannose-1-phosphatase, and permeases that are involved in the transport of mannose, or a derivative or a precursor thereof, into the cell. An agent is typically used to reduce the toxicity of the compound to the cells, and is typically a glucose derivative such as methyl-3-O-glucose or phloridzin. Transformed cells are identified without damaging or killing the non-transformed cells in the population and without co-introduction of antibiotic or herbicide resistance genes. As described in PCT International Publication No. WO 93/05163, in addition to the fact that the need for antibiotic or herbicide resistance genes is eliminated, it has been shown that the positive selection method is often far more efficient than traditional negative selection.

For expression of a nucleotide sequence of the presently disclosed subject matter in plant plastids, plastid transformation vector pPH143 (PCT International Publication WO 97/32011, example 36) can be used. The nucleotide sequence is inserted into pPH143 thereby replacing the protoporphyrinogen oxidase (Protox) coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

In another embodiment, a nucleotide sequence of the presently disclosed subject matter is directly transformed into the plastid genome. Plastid transformation technology is described in U.S. Pat. Nos. 5,451,513; 5,545,817; and 5,545,818; and in PCT International Publication No. WO 95/16783; and in McBride et al., 1994.

Another approach to transforming plant cells involves propelling inert or biologically active particles at plant tissues and cells. This technique is disclosed in U.S. Pat. Nos. 4,945,050; 5,036,006; and 5,100,792; all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue.

Transformation of most monocotyledon can include direct gene transfer into protoplasts using PEG or electroporation, and particle bombardment into callus tissue. Transformations can be undertaken with a single DNA species or multiple DNA species (i.e. co-transformation), and both these techniques are suitable for use with the presently disclosed subject matter. Co-transformation can have the advantage of avoiding complete vector construction and of generating transgenic plants with unlinked loci for the gene of interest and the selectable marker, enabling the removal of the selectable marker in subsequent generations, should this be regarded as desirable. However, a disadvantage of the use of co-transformation is the less than 100% frequency with which separate DNA species are integrated into the genome (Schocher et al., 1986).

Transformation of monocotyledons using Agrobacterium has also been disclosed. See WO 94/00977 and U.S. Pat. No. 5,591,616, both of which are incorporated herein by reference. See also Negrotto et al., 2000, Zhao et al., 2000, and also U.S. Pat. No. 6,369,298, which is incorporated herein by reference.

Once formed, transgenic plant cells can be placed in an appropriate selective medium for selection of transgenic cells, which are then grown to callus. Shoots are grown from callus and plantlets generated from the shoot by growing in rooting medium. The various constructs normally are joined to a marker for selection in plant cells. Conveniently, the marker can be resistance to a biocide (for example, an antibiotic including, but not limited to kanamycin, G418, bleomycin, hygromycin, chloramphenicol, herbicide, or the like). The particular marker used is designed to allow for the selection of transformed cells (as compared to cells lacking the DNA that has been introduced). Components of DNA constructs including transcription cassettes of the presently disclosed subject matter are prepared from sequences that are native (endogenous) or foreign (exogenous) to the host. As used herein, the terms “foreign” and “exogenous” refer to sequences that are not found in the wild-type host into which the construct is introduced, or alternatively, have been isolated from the host species and incorporated into an expression vector. Heterologous constructs contain in one embodiment at least one region that is not native to the gene from which the transcription initiation region is derived.

To confirm the presence of the transgenes in transformed cells and plants, a variety of assays can be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (enzyme-linked immunosorbent assays (ELISAs) and Western blots) or by enzymatic function; plant part assays, such as seed assays; and also by analyzing the phenotype of the whole regenerated plant, e.g., for disease or pest resistance.

DNA can be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The present disclosure may be better understood with reference to the Example, set forth below.

### Example

### Testing Procedures

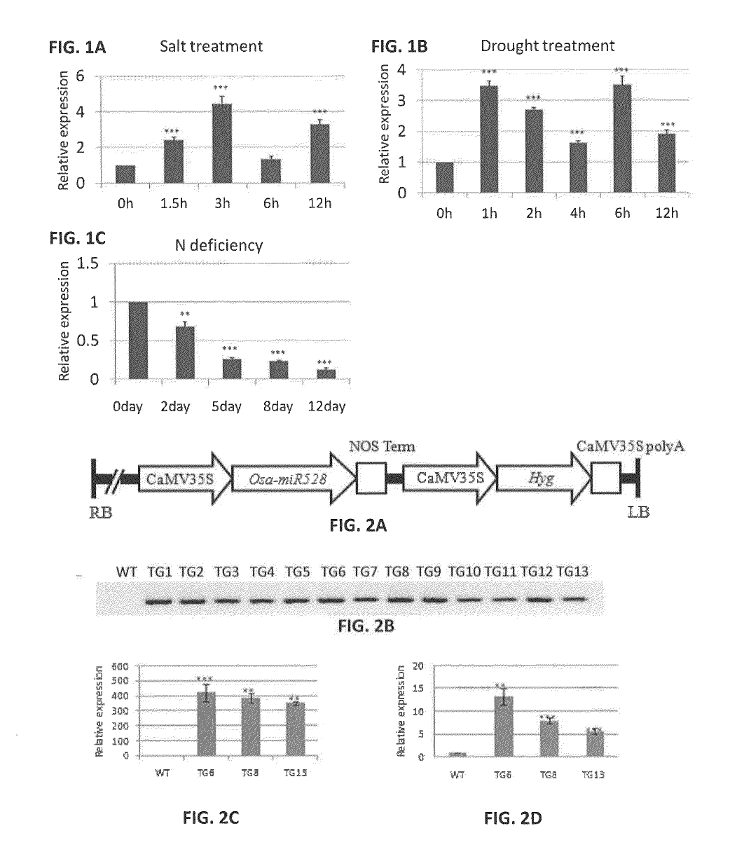

The full length of Osa-miR528 gene (SEQ ID NO: 1) (Os03g0129400) containing the precursor miR528 stem-loop structure (SEQ ID NO: 2) was isolated by PCR from rice (Oryza sativa) cDNA. The Osa-miR528 gene forward and reverse primer set was 5′-TCTAGAGATCAGCAGCAGCCACA-3′ (SEQ ID NO: 11) containing an Xbal recognition site and 5′-GTCGACGACCAAATAATGTGTTACTG-3′ (SEQ ID NO: 12) containing a SalI recognition site. PCR products were cloned into the binary vector pZH01, generating the Osa-miR528 overexpression gene construct, p35S-Osa-miR528/p35S-hyg. The construct (FIG. 1A) contained the cauliflower mosaic virus 35S (CaMV 35S) promoter driving Osa-miR528 which is linked to the CaMV 35S promoter driving the hyg gene for hygromycin resistance as a selectable marker. For subsequent plant transformation, the construct was transferred into Agrobacterium tumefaciens strain LBA4404.

Creeping bentgrass (Agrostis stolonifera L.) cultivar ‘Penn A-4’ (supplied by HybriGene) was used for plant transformation. Transgenic plants constitutively expressing Osa-miR528 were produced via Agrobacterium-mediated transformation of embryonic callus induced from mature seeds according to known methodology.

The regenerated transgenic plants overexpressing Osa-miR528 were transferred in commercial nutrient-rich soil (3-B Mix, Fafard) and initially maintained in the greenhouse with wild type (WT) controls at 27 CC during the light and 25° C. during the dark under long day conditions (16 h of light/8 h of dark).

To conduct the abiotic stress treatments, transgenics and WT plants were vegetatively propagated from tillers and grown in Cone-tainers™ (4.0×20.3 cm, Dillen Products), small pots (9.8×7 cm, Dillen Products), middle pots (15×10.5 cm, Dillen Products), or big pots (33×44.7 cm, Dillen Products) using silica sand. The plants were maintained in the growth room in a 14-h-light/8-h-dark photoperiod at 350-450 μmol/m2s light intensity provided by AgroSun Gold 1000 W sodium/halide lamps (Maryland Hydroponics). Temperature and humidity were maintained at 25° C./17° C. (light/dark), and 30%/60% (light/dark) respectively. Plants were watered every other day with 0.2 g/L 20:10:20 water-soluble fertilizer (Peat-Lite Special; The Scotts Company) and mowed every week to achieve uniform growth.

For salt stress treatments, plants grown in Cone-tainers™ and small pots were immersed in the 200 mM NaCl solution supplemented with 0.2 g/L water-soluble fertilizer. The salt solution was changed every other day. After nine-day salt treatments, shoots were harvested for further physiological analyses. Plants' recovery from salt treatment by watering 0.2 g/L water-soluble fertilizer every other day was documented by photography.

To test performances of WT and TG plants under different concentrations of nitrogen, plants grown in Cone-tainers™ were immersed in modified Murashige and Skoog (MS) nutrient solution containing 3 mM CaCl2.2H2O, 1.5 mM MgSO4.7H2O, 1.25 mM KH2PO4, 0.1 mM H3BO3, 0.1 mM MnSO4.4H2O, 0.1 mM ZnSO4.2O, 0.5 μM KI, 0.56 μM NaMO4.2H2O, 0.1 μM CuSO4.8H2O, 0.1 μM CoCl2.6H2O, 0.1 mM FeSO4.7H2O, 0.1 mM Na2EDTA.2H2O, and different nitrogen concentrations which were 0.4 mM, 2 mM, 10 mM or 50 mM. The nutrient solution was refreshed every week. Five weeks later, shoots were harvested for further physiological analyses.

For drought stress tests, plants in Cone-tainers™ and big pots were subjected to water withholding. One week and three weeks later, shoots were harvested from Cone-tainers™ and big pots separately for further analyses.

Plant genomic DNA was extracted from 30 mg of fresh leaves in 1.5 mL microcentrifuge tube using 2× cetyltrimethyl ammonium bromide (CTAB) buffer following a known protocol, Plant total RNA was isolated from 100 mg of fresh leaves using Trizol reagent (Invitrogen) following the manufacturer's protocol. First strand cDNA was synthesized from 2 μg of RNA with SuperScript III Reverse Transcriptase (Invitrogen) and oligo(dT) or gene specific primers. The semi-quantitative RT-PCR was conducted on 24 to 30 cycles based on its exponential phase. PCR products were separated by using 1.5% agarose gel electrophoresis and visualized as well as photographed with Gel-doc (Bio-Rad Laboratories).

Real-time RT-PCR was performed with 12.5 μL of iQ SYBR-Green Supermix (Bio-Rad Laboratories) per 25 μL reaction system. The green fluorescence signal was monitored on Bio-Rad iQ5 real-time detection system by using iQ5 Optical System Software version 2.0 (Bio-Rad Laboratories). AsACT1 (JX644005) and AsUBQ5 (JX570760) were used as endogenous controls. The relative changes of gene expression were calculated based on 2-ΔΔCT method [65], in which ΔΔCT=[(CT gene of interest−CT reference gene) control sample−(CT gene of interest−CT reference gene) treated sample].

Stem-loop RT-qPCR was performed according to Varkonyi-Gasic's protocol. The osa-miR528 stem-loop RT primer and PCR forward primer are 5′-GTCGTATCCAGTGCAGGGTCCGAGGTATTCGCACTGGATACGACCTCCTC-3′ (SEQ ID NO: 13) and 5′-GCAGTGGAA GGGGCATGCA-3″ (SEQ ID NO: 14) separately.

For Na+, K+, and Cl⁻ content measurement, WT and transgenic plant leaves were collected before and after two weeks of 200 mM NaCl solution treatment. For total nitrogen measurement, WT and transgenic plant leaves were collected after five weeks of different concentrations of nitrogen solution treatments, including 0 mM, 0.4 mM, 2 mM, and 10 mM. Fresh leaves collected for mineral content measurement were dried at 80° C. for 48 hours. 0.2 gram of each dried sample for total nitrogen measurement and 1 gram of each dried sample for Na⁺, K⁺, and Cl⁻ content measurement were determined according to standard protocols.

Plant leaf RWC was measured following known protocols. Briefly, plant leaves were harvested and fresh weight (FW) was measured. The material was then immersed in 20 mL of Millipore water overnight at 4° C. After weighing the turgid weight (TW), the leaves were dried at 80° C. for 24 hours for dry weight (DW) measurement. RWC was determined using the equation RWC=[(FW−DW)/(TW−DW)]×100%.

Leaf EL was measured according to known protocols. Briefly, 0.2 gram of plant leaves were harvested and immersed in 20 mL of Millipore water at 4° C. overnight. To determine the amount of ions released from leaf tissue, the initial conductance (Cᵢ) of the incubation solution was measured. To determine the total amount of ions in the leaf tissue, the maximum conductance (Cₘₐₓ) was measured after 30 minutes autoclaving and 24 hours shaking of the incubation solution containing leaves. EL was determined using the equation EL=(Cᵢ/Cₘₐₓ)×100%.

Two replicates of 100 mg of plant fresh leaves were collected under normal and stress treated conditions and stored at −80° C. for subsequent analyses. Plant chlorophyll a and b as well as proline contents were measured according to known protocols.

WT and TG initiating from the same amount of tillers were propagated in the same middle pot (15×10.5 cm, Dillen Products). Four weeks later, from the top of tillers, the second and third internodes and fully expanded leaves were collected and immersed in formalin-acetic alcohol fixation which contains 50% of 100% ethanol, 10% of 37% formaldehyde solution and 5% glacial acetic acid for 48 hours at room temperature. After fixation, plant tissues were dehydrated with a series of graded ethanol from 70% to 100%, followed by paraffin wax infiltration. Tissues were then embedded in paraffin blocks. When paraffin solidified, blocks were ready to process section using the rotary microtome (RM 2165, Leica). Sections were stained using toluidine blue and observed under stereo microscope (MEIJI EM-5). Photographs were taken using 35 mm SLR camera body (Canon) connected to the microscope. Scale bars were added to photographs using ImageJ.

### Results

miR528 was examined in a perennial species, creeping bentgrass, to determine if it is involved in the response to abiotic stress. Wild type turfgrass plants were treated with 200 mM NaCl, water withholding, and N deficiency. Quantitative stem-loop RT-PCR analyses (FIG. 1) indicate that miR528 was regulated by salt (FIG. 1A), drought (FIG. 1B), and N starvation (FIG. 1C). The relative changes of gene expression were calculated based on 2^{-ΔΔCT} method. AsActin was used as an endogenous control. Data are presented as average of three technical replicates, and error bars represent ±SE. Asterisks (** or***) indicate a significant difference of expression levels between untreated and each abiotic stress treated WT plants at P<0.01 or 0.001 by Student's t-test.

The miR528 overexpression construct was produced and introduced into the genome of WT creeping bentgrass through Agrobacterium tumefaciens mediated transformation. The full length of Osa-miR528 (Os03g0129400) (SEQ ID NO: 1) containing pre-miR528 stem-loop structure (SEQ ID NO: 2) was amplified through PCR, and then cloned into the binary vector pZH01, generating the Osa-miR528 overexpression gene construct, p35S-Osa-miR528/p35S-hyg. As shown in FIG. 2A the Osa-miR528 gene was under the control of Cauliflower Mosaic Virus (CaMV) 35S promoter and linked to the hygromycin resistance gene, Hyg, driven by CaMV 35S promoter. To select positive transgenic plants containing miR528 overexpression constructs, Hyg gene was amplified with genomic DNA of regenerated plants after transformation. Through PCR analysis, 13 transgenic lines in total were obtained (FIG. 2B), which were morphologically indistinguishable. Three transgenic lines, TG6, TG8 and TG13 were chosen for further characterization on the aspects of plant development and stress response. To detect whether the primary sequence of Osa-miR528 (pri-miR528) had been integrated into the host genome at RNA level, quantitative Reverse transcription (RT) PCR analysis was conducted to compare the expression levels of pri-miR528 between WT control and three transgenic lines. The result indicated that transcripts of pri-miR528 were significantly higher in three transgenic lines than in WT controls (FIG. 2C). To determine whether pri-miR528 could process into miR528 mature sequence successfully, quantitative stem-loop RT-PCR analysis was carried out. The expression levels of mature Osa-miR528 in three transgenic lines were significantly high in comparison with WT plants (FIG. 2D), suggesting that primary sequence of Osa-miR528 form rice can be processed properly in creeping bentgrass. The relative changes of gene expression were calculated based on 2-ΔΔCT method. AsActin was used as an endogenous control. Data are presented as average of three technical replicates, and error bars represent ±SE. Asterisks (** or ***) indicate a significant difference of expression levels between WT and each transgenic line at P<0.01 or 0.001 by Student's t-test.

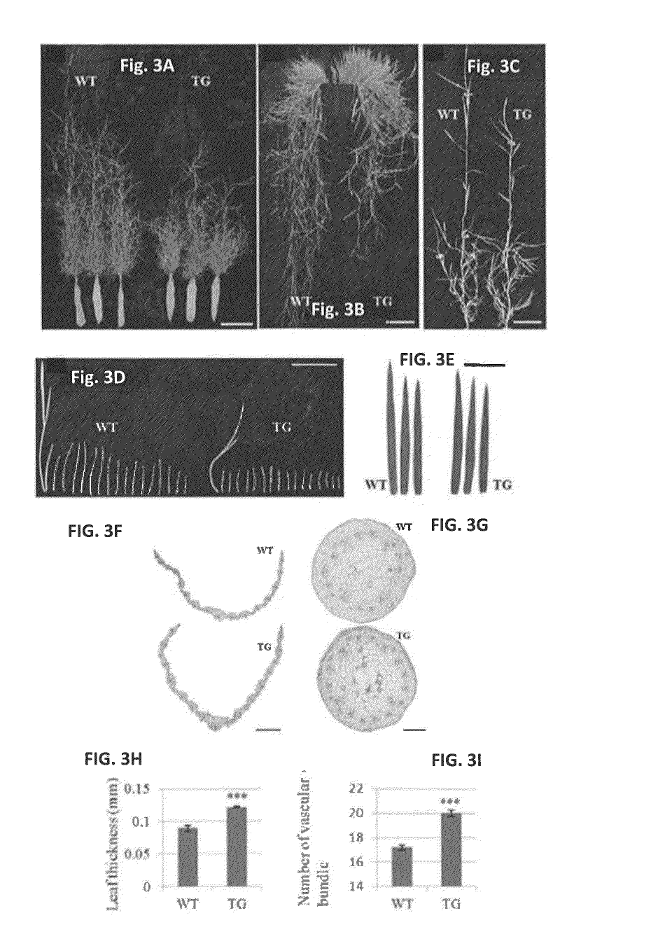

To determine the involvement of miR528 in plant development, we analyzed WT and TG plants initiated from a single tiller in pure sand. TG plants produce significantly more, but shorter tillers than WT controls (FIG. 3A, FIG. 3B, FIG. 3C, and FIG. 4A), especially at the later developmental stage (ten-week-old, FIG. 4A). However, no significant difference in the total numbers of shoots, the primary and secondary tillers from a crown and internodes was observed between WT and TG plants at the later developmental stages (60- and 90-day-old, FIG. 48). The developmental changes observed in TG plants were further confirmed by comparing WT and Osa-miR528 TG plants grown in the same pot filled with soil (FIG. 3B). In addition, TG plants exhibited more upright tiller growth than WT controls (FIG. 3B).

To further study what causes the reduced tiller length in transgenics, we analyzed the average length and number of the internodes of the representative tillers from WT and TG plants (FIG. 3D). We found that the total numbers of the internodes in WT and TG tillers are similar, whereas the average length of the internodes from each tiller in TG plants is significantly reduced compared with WT controls (FIG. 4C). For FIGS. 4A, 4B, and 4C, data are presented as average, and error bars represent ±SE. Asterisks (*, **, or ***) indicate a significant difference of shoot number, tiller number, or internodes length between WT and each transgenic line at P<0.05, 0.01, or 0.001 by Student's t-test.

TG and WT leaves and stems were also compared at the cellular level via histological analysis (FIG. 3E, FIG. 3F, FIG. 3G). Transgenic leaves were significantly thicker than WT leaves (FIG. 3H) and the number of the stem vascular bundles was significantly increased in transgenics compared to that in WT controls (FIG. 3I).

The potential impact of miR528 on plant growth was investigated by measuring the shoot and root biomass of the ten-week-old WT and TG plants initiated from a single tiller, and the weekly clipping weight thereafter for continuous four weeks. Our statistical analyses indicate no significant difference in biomass accumulation between TG and WT plants (FIG. 5A-FIG. 5F).

The impact of TG on plant growth rate was investigated by measuring shoot and root biomass of ten-week-old WT and TG plants initiating from single tiller. Statistical analyses indicated that there was no significant difference of shoot and root biomass including both fresh and dry weight between WT controls and transgenics (FIG. 5A-FIG. 5D). The growth rate of WT and TG plants was also evaluated through clipping collection. Fully developed WT and TG plants starting from the same amount of tillers were trimmed to the same height every week. Clipping was collected and weighed for a continuous four weeks. FIG. 5E and FIG. 5F illustrate the accumulated fresh or dry weight of clipping from one to four weeks. After four weeks clipping collection, there was no significant difference between WT controls and three transgenic lines, which further confirms the biomass discussed above. Data are presented as average, and error bars represent ±SE. Asterisk (*) indicates a significant difference of shoot or root biomass between WT and each transgenic line at P<0.05 by Student's t-test.

To investigate if constitutive expression of Osa-miR528 by transgenic creeping bentgrass will enhance its resistance to salt stress, the performance of WT and TG plants after salt treatment was evaluated. Fully developed WT plants and two TG lines initiating from the same amount of tillers were trimmed to be uniform before the test (FIG. 6A), and 200 mM NaCl was applied for 14 days followed by recovery. During the recovery stage, both WT and TG plant leaves displayed light green and senescence phenomenon in comparison with those leaves before the treatment, but WT plants had more severe responses than those of transgenics (FIG. 6B and FIG. 6C).

Water stress will damage plant cell membrane and turgidity. Therefore, the maintenance of cell membrane integrity and water status are considered major components in plant salt stress tolerance. To investigate the degree of cell membrane injury between WT and TG plants under salt stress, the plant electrolyte leakage (EL) was measured. Under normal growth conditions, there was no significant difference of EL between WT and two TG lines. After nine days of salt stress treatment, EL value in WT and two TG lines were all increased as compared to that before the treatment, but the EL value of WT was significant higher than that of two transgenic lines (FIG. 6D), indicating that TG plants had better capability to maintain cell membrane integrity than that of WT controls under salt stress conditions. To compare the water status in WT and TG plants, the relative water content (RWC) was measured before and after the stress treatment. Similar RWC was displayed under normal growth conditions (FIG. 6E). When plants were subjected to salinity for nine days however, TG plants had significantly higher RWC than that of WT controls (FIG. 6E), which implied that TG plants had improved ability to retain water under salinity stress in comparison with WT controls.

Besides cell membrane integrity and turgidity, leaf chlorophyll content was also affected under salt stress, most likely due to the destruction of chlorophyll pigment protein complex, the degrading of chlorophyll enzyme chlorophyllase, and the interference on the synthesis of chlorophyll structural components. The salt stress treated WT and TG plants displayed less green compared to those non-stress treated plants (FIG. 6A and FIG. 6B), which was in agreement with previous studies.

The chlorophyll a, chlorophyll b and total chlorophyll concentrations in WT and TG plants were measured before and after NaCl stress. Under normal growth conditions, there was no significant difference of chlorophyll contents between WT and TG plants, while all three transgenic lines showed significantly higher chlorophyll contents than that of WT controls (FIG. 7A, FIG. 7B, FIG. 7C), suggesting the possible role of transgenics in improving photosynthesis system and contributing to enhanced salt stress resistance. Data are presented as average (n=5), and error bars represent ±SE. Asterisks (*, ** or ***) indicates a significant difference of chlorophyll contents between WT and each transgenic lines at P<0.05, 0.01 or 0.01 by Student's t-test.

Proline is essential for plant primary metabolism under salt stress. It plays a molecular chaperone role in buffering the pH of the cytosolic redox status within the cell and in ROS scavenging. Before the salt stress, proline contents in WT and TG plants were similar; however, proline contents increased dramatically in both WT controls and TG plants after salinity stress (FIG. 8). In addition, transgenics accumulated significantly higher proline contents than controls, implying enhanced ROS detoxification capacity under osmotic stress in comparison with WT controls.

Salt stress imposes ionic imbalance and osmotic stress on plants due to elevated Na⁺ levels around plant roots. To compare the Na⁺ uptake in WT and Osa-miR528 TG plants, Na⁺ relative contents were measured. Before the salt stress, three transgenic lines have significantly higher Na⁺ accumulation in shoots than WT controls, while they have similar Na⁺ levels in roots (FIG. 9A). After the salt treatment, WT and TG plants have similar Na⁺ contents in shoots and roots (FIG. 9B).

Potassium (K) plays an essential role in diverse physiological processes including turgor adjustment, stomata movement, cell elongation, and activation of more than 50 cytoplasmic enzymes. Salinity also affects K⁺ homeostasis, because Na⁺ competes with K⁺ for binding sites during enzymatic reactions and protein syntheses in the cytoplasm where K⁺ functions as a co-factor in these processes. Our result shows that K⁺ relative contents in WT and TG shoots are similar or slightly higher in TG shoots before salt stress (FIG. 9C). After salinity treatment, interestingly, transgenics maintain their shoot K⁺ level, whereas, the K⁺ levels in WT shoots drop dramatically, becoming significantly lower than that in transgenic shoots (FIG. 9D). Transgenics also contain higher K⁺ in roots than WT plants, although the difference is insignificant (FIG. 9C and FIG. 9D).

One of the key elements in plant salinity tolerance is the capacity of maintaining a high K⁺:Na⁺ ratio. Under normal growth conditions, WT shoots have significantly higher K⁺:Na⁺ ratio than transgenics due to their lower Na⁺ contents than transgenic shoots (FIG. 9E). After salt stress treatment, however, K⁺:Na⁺ ratios of shoots and roots are both significantly higher in transgenics than in WT controls (FIG. 9F), FIG. 9G shows that under salt stress, transgenics are capable of maintaining similar shoot K⁺ levels to non-stressed conditions compared to WT controls. However, K⁺ levels in both WT and TG roots decrease dramatically although transgenic roots have higher K⁺ contents than WT controls under non-stressed conditions (FIG. 9H). Data are presented as means (n=3), and error bars represent ±SE. Asterisks (*, **, or ***) indicate significant differences of K⁺ content, Na⁺ content, or K⁺:Na⁺ ratio between WT and each transgenic line at P<0.05, 0.01, or 0.001 by Student's t-test.

Differences of Na⁺ and K⁺ contents between WT and TG plants imply that miR528 might mediate the concerted action of ion transport systems. To investigate the underlying mechanism of miR528-mediated ion transport, K transporter genes in creeping bentgrass were identified and their expression were analyzed in TG and WT plants. Previous studies indicate that there are mainly seven gene families involved in K⁺ uptake, of which, functionally characterized genes encoding K permeable channels and K transporters were selected for further study. AsHAK5 from KP/HAK/KT transporter family is successfully amplified in creeping bentgrass and found to be up-regulated in TG leaves and roots compared to WT controls (FIG. 18), suggesting that constitutive expression of miR528 leads to enhanced K transporter activity and contribute to the increased K⁺ uptake and enhanced capacity of maintaining K⁺ homeostasis in TG plants.

Plants have evolved stress tolerance strategy of ROS detoxification via increasing antioxidant enzyme activity. In addition, miR528 predicted targets are involved in oxidation-reduction. In order to understand how these enzymes involve in plant salt stress response in both WT and TG plants, CAT and AAO enzyme activity was measures. CAT catalyzes the decomposition of hydrogen peroxide to water and oxygen. Our results indicate that transgenic plants have significantly higher CAT activity than that of WT controls under both normal and salt stress conditions (FIG. 10A). AAO catalyze the reaction of ascorbate (AsA) oxidation, which will reduce the redox status of AsA. AsA is involved in maintaining equilibrium of ROS and help cells avoid oxidative stress. Under salt stress, transgenic plants showed significantly lower AAO activity than that of WT plants (FIG. 10B), suggesting that transgenics have more AsA under redox status and contributing to better elimination of ROS.

To examine the responses of WT and TG plants under nitrogen deficiency conditions, the optimum nitrogen concentration was determined for creeping bentgrass at first by applying MS nutrient solutions containing 2 mM, 10 mM, or 40 mM nitrogen to two-month-old WT and TG plants initiating from the same amount of tillers (FIG. 11A). Four weeks later WT controls and three transgenic lines had a rapid growth under 10 mM nitrogen solutions compared with 2 mM and 40 mM nitrogen solutions (FIG. 11B), so 10 mM became the optimum nitrogen level in our experiment and used for further analysis. Plants treated with 2 mM nitrogen solution displayed lighter green than that of plants treated with 10 mM and 40 mM nitrogen solutions (FIG. 11B), because nitrogen starvation contributes to the degradation of chlorophyll for nutrient recycling. Result also showed that excess nitrogen levels of 40 mM reduced plant growth (FIG. 11B), due to the decreased uptake of other nutrient elements, like phosphate and potassium. The statistical analyses of shoot fresh and dry weight in WT controls and two representative transgenic lines indicated that both WT and TG plants reached their highest growth rate with 10 mM nitrogen treatment; while they had the least biomass with 2 mM nitrogen treatment (FIG. 11E and FIG. 11F). In addition, WT and TG plants had similar shoot biomass with 10 mM nitrogen treatment, but transgenics had more shoot fresh and dry weight under in both low nitrogen (2 mM) and high nitrogen (40 mM) conditions (FIG. 11E and FIG. 11F). Besides biomass difference, we also observed wilting leaf tips only in WT plants under all of three nitrogen nutrient solution treatments (FIG. 11C). Data are presented as average (n=4), and error bars represent ±SE. Asterisks (*, or **) indicates a significant difference of biomass value between WT and transgenic plants at P<0.05 or 0.01 by Student's t-test.

The total nitrogen content was compared in WT and TG plants under N-starved (2 mM), N-sufficient (10 mM), and N-excess (40 mM) conditions. The result indicates that the higher concentration of nitrogen solution applied, the more total nitrogen content plants contain (FIG. 12A, FIG. 12B). However, there was no significant difference between WT and three transgenic lines under N-starved, N-sufficient and N-excess conditions. The total nitrogen content was measured as the percentage of the unit weight (FIG. 12A), implying that TG shoots accumulated more total nitrogen under N-starved and N-excess conditions for the reason that TG plants had more shoot biomass than WT controls under both conditions. WT & transgenic turfgrass overexpressing Osa-miR528 were applied with different concentrations (2 mM, 10 mM, 40 mM) of nitrogen solutions for 4 weeks. Shoots total nitrogen was measured after the treatment. Data are presented as average (n=4), and error bars represent ±SE.

Nitrogen deficient plants were observed to have a lighter green color than plants under N-sufficient and N-excess conditions. To quantitatively measure differences of chlorophyll between N-starve and N-sufficient plants, as well as between WT and TG plants, we detected the chlorophyll contents. In comparison with N-sufficient plants, plants under nitrogen deficiency conditions (0.4 mM and 2 mM) showed low total chlorophyll content including both chlorophyll a and b, especially under 0.4 mM nitrogen condition (FIG. 13A, FIG. 13B, FIG. 13C). Additionally, WT and TG plants had similar chlorophyll content under N-sufficient conditions. TG plants, however, showed significant higher chlorophyll content than WT controls under N-starved conditions (FIG. 13A, FIG. 13B, FIG. 13C), indicating a less degree of chlorophyll degradation and relatively increased photosynthetic capability in TG plants under nitrogen deficiency conditions. Data are presented as average (n=5), and error bars represent ±SE. Asterisks (*, **, or **) indicates a significant between WT and transgenic plants at P<0.05, 0.01 or 0.001 by Student's t-test.

To investigate what causes the enhanced NUE, we examined the transcript levels of key enzymes in N assimilation pathway in WT and transgenic creeping bentgrass. The enzymes include nitrate reductase (NR), NiR, glutamine synthetase (GS), and glutamate synthase (GOGAT). As shown in FIG. 14A, the expression of AsNiR, but not AsNR, AsGS, or AsGOGAT, is significantly up-regulated in transgenic plants in comparison with WT controls. Consistently, the enzyme activity of the NiR is also significantly higher in transgenic plants than in WT controls before and after N starvation treatment although its activity increases in both WT and TG plants in response to N starvation (FIG. 14B). The error bars represent ±SE. Asterisks (*,** or ***) indicate significant differences of expression levels or enzyme activities between WT and TG plants at P<0.05, 0.01 or 0.001 by Student's t-test.

To understand the underlying molecular mechanisms of miR528-mediated plant response to salinity and N deficiency, we sought to identify putative targets of miR528 in creeping bentgrass. Currently, only SsCBP1, a copper ion binding domain-containing protein is experimentally confirmed as the target of miR528 in sugarcane. In rice, Os06g37150 encoding AAO is validated as the target of miR528 through a high-throughput degradome sequencing approach. To identify its targets in creeping bentgrass, a plant small RNA target analysis tool (psRNA Target) was applied to predict targets in rice genome. Eleven putative targets were recognized in rice, among which partial fragments of four genes were successfully amplified in creeping bentgrass based on the sequence similarity to rice. Genes encoding AAO and CBP1 show decreased expression in TG plants (FIG. 15A, FIG. 15B), indicating that they might be targets of miR528 in creeping bentgrass. Targeting site of miR528 in AsCBP1 was detected in its open reading frame. Interestingly, target site of miR528 cannot be detected in the coding region of AsAAO, RACE analysis showed that it is located in the 3′UTR at 26 nt to 45 nt region. The descriptions, functions and corresponding orthologues in rice and Arabidopsis of AsAAO and AsCBP1 are listed in FIG. 15C.

AsAAO functions in oxidation-reduction, implying its important role in plant abiotic stress response. AsCBP1 encodes a cupredoxin superfamily protein. Proteins from this family function in oxidation homeostasis and electron transfer reactions, which are involved in photosynthesis, respiration, cell signaling, and numerous reactions of oxidases and reductases. To investigate whether AsAAO and AsCBP1 respond to salt stress and N deficiency conditions, we conducted semi-quantitative RT-PCR analysis to examine their expression profiles under salt (FIG. 19A) and N deficiency (FIG. 19B) treatments. FIG. 19A, FIG. 19B show that AsAAO are too low to be detected in leaf and root tissues before stress test. However, its expression levels in leaf are induced dramatically under salt treatment and gradually increase in leaves (FIG. 19A). When plants are exposed to N deficiency, the expression of AsAAO is significantly induced five days after treatment, and then declined thereafter (FIG. 19B). Interestingly, AsAAO expression is too low to be detected in root tissues under both normal and stressed conditions by semi-quantitative RT-PCR (FIG. 19A, FIG. 19B). When plants are exposed to salt stress, AsCBP1 displays similar expression levels in leaf tissues in comparison with the normal growth conditions; however, its transcript levels are gradually increased in root tissues (FIG. 19A). During N starvation, AsCBP1 is induced eight days after treatment in leaf tissues, while its expression is gradually induced from 0-day to 5-day treatment, and then declined thereafter in root tissues (FIG. 19B).

The important role of miRNAs has been gradually recognized in the complex stress response network. In order to determine if miR528 has crosstalk with other stress responsive miRNAs, real-time PCR was conducted to analyze the expression levels of miR156 and its target genes AsSPL3, AsSPL16 in WT turfgrass and transgenic plants overexpressing osa-miR528. It was found that miR156 expression levels was decreased in three TG lines, and its target genes AsSPL3, AsSPL16 were upregulated in TG plants (FIG. 16A, FIG. 16B). SPLs are involved in control grass brunching. Increased transcript levels of SPL genes might contribute to the increased tiller number in transgenics.

The accumulation of miR528 is elevated during salt stress (FIG. 1A), which represses the transcription of its targets AsAAO and AsCBP1 (FIG. 15). Both targets respond to salinity and N starvation (FIG. 19) and are suggested to mediate the oxidation homeostasis and thus preventing damage to cellular components. Besides the direct targets of miR528, genes involved in other signaling pathways also contribute to the enhanced salt stress tolerance. A high-affinity K transporter AsHAK5, induced in transgenic creeping bentgrass overexpressing miR528 (FIG. 18), is critical for maintaining the K⁺ homeostasis during normal and salinity conditions. Moreover, miR528 induces the activity of CAT, and therefore maintaining the ROS homeostasis under abiotic stress. In addition to functional proteins, miR528 also positively regulates AsNAC60 (FIG. 17D), which is a creeping bentgrass orthologue of a salt stress-induced transcription factor, suggesting the importance of AsNAC60 in miR528-mediated salt stress tolerance in creeping bentgrass. MiR528 is gradually repressed during N deficiency (FIG. 1C), and therefore releasing the inhibition of its targets, which contribute to the oxidation homeostasis. AsNiR, a key enzyme in the process of N assimilation pathway, is positively regulated by miR528 (FIG. 14B). The enhanced NUE is presumably attributed to the increased AsNiR activity. MiRNAs are suggested to serve as the master regulators in the complex regulatory network of plant response to abiotic stress. The impact of miR528 on the expression of other stress-related miRNAs observed in this study (FIG. 17A, FIG. 17B, FIG. 17C) suggests coordinated interactions of multiple stress regulators, and thereby leading to the enhanced salt and N deficiency tolerance.

The hypothetical model of miR528-mediated plant abiotic stress response pathway (FIG. 20) allows development of novel molecular strategies to genetically engineer crop species for enhanced environmental stress tolerance. As illustrated in FIG. 20, MiR528 is induced during salinity stress, but down-regulated under N deficiency. MiR528 mediates plant abiotic stress responses through directly repressing the expression of its targets AsAAO and AsCBP1, which regulate the oxidation homeostasis during abiotic stresses. In addition, miR528 positively regulates AsNAC60, AsHAK5, AsNiR and the gene encoding antioxidant enzyme CAT, which leads to the enhanced tolerance to salinity stress and N deficiency. Furthermore, expression levels of other stress-related miRNAs are negatively regulated by miR528, suggesting that different miRNAs form a regulatory network to coordinately integrate various signals in response to plant abiotic stress.

It will be appreciated that the foregoing examples, given for purposes of illustration, are not to be construed as limiting the scope of this disclosure. Although only a few exemplary embodiments of the disclosed subject matter have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A transgenic plant cell including a recombinant nucleic acid molecule, the recombinant nucleic acid molecule comprising a polynucleotide encoding miR528 operatively associated with a promoter.

2. The transgenic plant cell of claim 1, wherein the polynucleotide encoding miR528 comprises SEQ ID NO.: 1.

3. The transgenic plant cell of claim 1, wherein the polynucleotide encoding miR528 comprises SEQ ID NO.: 2.

4. A transgenic plant comprising the transgenic plant cell of claim 1.

5. The transgenic plant of claim 4, wherein the transgenic plant is a turfgrass plant.

6. A crop comprising a plurality of the transgenic plants of claim 4.

7. A transgenic seed comprising the transgenic plant cell of claim 1.

8. A transgenic plant cell including a recombinant nucleic acid molecule, the recombinant nucleic acid molecule comprising a polynucleotide that is antisense to only a portion of consecutive nucleotides of the sequence of SEQ ID NO: 3, SEQ ID NO: 4, SEQ ID NO: 5 or SEQ ID NO: 6 or comprising a nucleotide sequence that encodes only a portion of consecutive nucleotides of SEQ ID NO: 3, SEQ ID NO: 4, SEQ ID NO: 5, or SEQ ID NO: 6, which when expressed produces an antisense nucleotide sequence, wherein a plant expressing the antisense nucleotide sequence exhibits increased tolerance to abiotic stress as compared to a plant lacking the recombinant nucleotide.

9. A transgenic plant comprising the transgenic plant cell of claim 8.

10. The transgenic plant of claim 9, wherein the transgenic plant is a turfgrass plant.

11. A crop comprising a plurality of the transgenic plants of claim 9.

12. A transgenic seed comprising the transgenic plant cell of claim 8.

13. A method for producing a plant, the method comprising:
transforming a plant cell with a recombinant nucleic acid molecule, the recombinant nucleic acid molecule comprising a nucleotide that encodes miR528 operative associated with a promoter; and generating a transgenic plant from the transformed plant cell.

14. The method of claim 13, wherein the nucleotide that encodes miR528 comprises SEQ ID NO: 1 or SEQ ID NO: 2.

15. The method of claim 13, wherein the transgenic plant exhibits increased tolerance to abiotic stress as compared to a wild type plant of the same species.

16. The method of claim 15, wherein the abiotic stress is water stress and/or nitrogen deficiency.

17. The method of claim 13, wherein the transgenic plant exhibits shorter internodes, more tillers, or more upright growth as compared to a wild-type plant of the same species.

18. The method of claim 13, wherein the plant is a turfgrass.
